(19) United States
(12) Patent Application Publication
(10) Pub. No.: US 2008/0248545 A1
(43) Pub. Date: Oct. 9, 2008

(54) Methods for Generating Novel Stabilized Proteins

(75) Inventors: Frances H. Arnold, Pasadona, CA (US); Yougen Li, Lawrenceville, NJ (US)

Correspondence Address: Gavrilovich Dodd & Lindsey LLP;Joseph R. Baker, APC, 8052 Avenida Secreto, Carlsbad, CA, 92009, US

(73) Assignee: THE CALIFORNIA INSTITUTE OF TECHNOLOGY, Pasadena, CA (US)

(21) Appl. No.: 12/024,515

(22) Filed: Feb. 1, 2008

Related U.S. Application Data
(60) Provisional application No. 60/899,120, filed on Feb. 2, 2007.
(60) Provisional application No. 60/900,229, filed on Feb. 8, 2007.
(60) Provisional application No. 60/918,528, filed on Mar. 16, 2007.

(51) Int. Cl. C12N 9/00; C07K 14/00; C12N 15/11; C12N 15/00; C12N 5/06

(52) U.S. Cl. 435/183; 530/350; 536/23.1; 435/320.1; 435/325

(57) ABSTRACT

The disclosure provides methods for identifying and producing stabilized chimeric proteins.

### CROSS REFERENCE TO RELATED APPLICATIONS

The application claims priority under 35 U.S.C. §119 to U.S. Provisional Application Ser. No. 60/899,120, filed Feb. 2, 2007, 60/900,229, filed Feb. 8, 2007; and 60/918,528, filed, Mar. 16, 2007 the disclosures of which are incorporated herein by reference.

### STATEMENT REGARDING FEDERALLY SPONSORED RESEARCH

The U.S. Government has certain rights in this invention pursuant to Grant No. GM068664 awarded by the National Institutes of Health and Grant No. DAAD19-03-0D-0004 awarded by ARO—US Army Robert Morris Acquisition Center.

### FIELD OF THE INVENTION

The invention relates to biomolecular engineering and design, including methods for the design and engineering of biopolymers such as proteins and nucleic acids.

### BACKGROUND

A repertoire of stable proteins that can be further refined for research, industry and medical use is important.

The disclosure provides chimeric polypeptides of P450. The disclosure provides a polypeptide selected from the group consisting of: (a) a polypeptide comprising sequences from CYP102A1 (“1”), A2 (“2”) and A3 (“13”) having the general formula 21122333, from N-terminus to C-terminus; (b) a polypeptide comprising sequences from CYP102A1 (“1”), A2 (“2”) and A3 (“3”) having the general formula 21322233, from N-terminus to C-terminus; and (c) a polypeptide comprising sequences from CYP102A1 (“1”), A2 (“2”) and A3 (“3”) having the general formula 22313333, from N-terminus to C-terminus, and wherein the polypeptide has a CO-binding peak at 450 nm. In one aspect, the polypeptide comprises a first peptide segment comprising about 64 to 68 amino acids having at the C-terminus of the first peptide segment a sequence E(S or E or K)RFD (SEQ ID NO:4), a second peptide segment comprising about 56 to 60 amino acids having at the C-terminus of the second peptide segment a sequence K(G or D)YH(A or E or S) (SEQ ID NO:5), a third peptide segment comprising about 42 to 46 amino acids having at the C-terminus of the third peptide segment a sequence GFNYR (SEQ ID NO:6), a fourth peptide segment comprising about 48 to 52 amino acid having at the C-terminus of the fourth peptide segment a sequence (D or S)LVD(K or S or R) (SEQ ID NO:7), a fifth peptide segment comprising about 50 to 54 amino acids having at the C-terminus of the fifth peptide segment a sequence HETTS (SEQ ID NO:8), a sixth peptide segment comprising about 58 to 62 amino acids having at the C-terminus of the sixth peptide segment a sequence PTAPA (SEQ ID NO:9), a seventh peptide segment comprising about 74 to 78 amino acids having at the C-terminus of the seventh peptide segment a sequence G(Q or M)QFA (SEQ ID NO:10), an eighth peptide segment comprising a sequence extending from the C-terminus of the seventh peptide segment to the C-terminus of the heme domain of a P450 BM3 of SEQ ID NO:1, 2, or 3, or the C-terminus of a P450 BM3 of SEQ ID NO:1, 2, or 3, and wherein the polypeptide has a CO-binding peak at 450 nm. The polypeptide of claim 1 or 2, wherein the polypeptide of (a) comprises from N-terminus to C-terminus SEQ ID NO:2 from amino acid residue 1 to about x1, SEQ ID NO:1 from about amino acid residue x1 to about x2, SEQ ID NO:1 from about amino acid residue x2 to about x3, SEQ ID NO:2 from about amino acid residue x3 to about x4, SEQ ID NO:2 from about amino acid residue x4 to about x5, SEQ ID NO:3 from about amino acid residue x5 to about x6, SEQ ID NO:3 from about amino acid residue x6 to about x7, SEQ ID NO:3 from about amino acid residue x7 to about x8; wherein x1 comprises residue 63, 64, 65, 66, or 67 of SEQ ID NO:2; x2 comprises residue 120, 121, 122, 123, or 124 of SEQ ID NO:1; x3 comprises residue 164, 165, 166, 167 or 168 of SEQ ID NO:1; x4 comprises residue 215, 216, 217, 218 or 219 of SEQ ID NO:2; x5 comprises residue 268, 269, 270, 271, or 272 of SEQ ID NO:2; x6 comprises residue 328, 329, 330, 331, or 332 of SEQ ID NO:3; x7 comprises residue 404, 405, 406, 407, or 408 of SEQ ID NO:3; and x8 comprises a residue corresponding to the C-terminus of the heme domain of CYP102A3 or the C-terminus of CYP102A3 of SEQ ID NO:3, and wherein the polypeptide has a CO-binding peak at 450 nm. In yet another aspect, the polypeptide of (b) comprises from N-terminus to C-terminus SEQ ID NO:2 from amino acid residue 1 to about x1, SEQ ID NO:1 from about amino acid residue x1 to about x2, SEQ ID NO:3 from about amino acid residue x2 to about x3, SEQ ID NO:2 from about amino acid residue x3 to about x4, SEQ ID NO:2 from about amino acid residue x4 to about x5, SEQ ID NO:2 from about amino acid residue x5 to about x6, SEQ ID NO:3 from about amino acid residue x6 to about x7, SEQ ID NO:3 from about amino acid residue x7 to about x8; wherein x1 comprises residue 63, 64, 65, 66, or 67 of SEQ ID NO:2; x2 comprises residue 120, 121, 122, 123, or 124 of SEQ ID NO:1; x3 comprises residue 164, 165, 166, 167 or 168 of SEQ ID NO:3; x4 comprises residue 215, 216, 217, 218 or 219 of SEQ ID NO:2; x5 comprises residue 268, 269, 270, 271, or 272 of SEQ ID NO:2; x6 comprises residue 328, 329, 330, 331, or 332 of SEQ ID NO:2; x7 comprises residue 404, 405, 406, 407, or 408 of SEQ ID NO:3; and x8 comprises a residue corresponding to the C-terminus of the heme domain of CYP102A3 or the C-terminus of CYP102A3 of SEQ ID NO:3, and wherein the polypeptide has a CO-binding peak at 450 nm. In yet a further aspect, the polypeptide of (c) comprises from N-terminus to C-terminus SEQ ID NO:2 from amino acid residue 1 to about x1, SEQ ID NO:2 from about amino acid residue x1 to about x2, SEQ ID NO:3 from about amino acid residue x2 to about x3, SEQ ID NO:1 from about amino acid residue x3 to about x4, SEQ ID NO:3 from about amino acid residue x4 to about x5, SEQ ID NO:3 from about amino acid residue x5 to about x6, SEQ ID NO:3 from about amino acid residue x6 to about x7, SEQ ID NO:3 from about amino acid residue x7 to about x8; wherein x1 comprises residue 63, 64, 65, 66, or 67 of SEQ ID NO:2; x2 comprises residue 121, 122, 123, 124, or 125 of SEQ ID NO:2; x3 comprises residue 164, 165, 166, 167 or 168 of SEQ ID NO:3; x4 comprises residue 214, 215, 216, 217 or 218 of SEQ ID NO:1; x5 comprises residue 268, 269, 270, 271, or 272 of SEQ ID NO:3; x6 comprises residue 328, 329, 330, 331, or 332 of SEQ ID NO:3; x7 comprises residue 404, 405, 406, 407, or 408 of SEQ ID NO:3; and x8 comprises a residue corresponding to the C-terminus of the heme domain of CYP102A3 or the C-terminus of CYP102A3 of SEQ ID NO:3, and wherein the polypeptide has a CO-binding peak at 450 nm.

The disclosure also provides polypeptides as set forth in Tables 1, 3, 4, 5, and 6 using the ternary system numerical identifiers.

The disclosure also provides a polynucleotide encoding any of the polypeptides above, as well as vectors and host cell comprising such polynucleotides.

The disclosure also provides an enzyme extract comprising a polypeptide produced from the host cells. Such enzymes are useful to catalyze reactions known in the art for P450 BM3 enzymes.

The sequences and thermostabilities of cytochrome P450 proteins assembled by structure-guided SCHEMA recombination were determined in order to identify relationships that would allow prediction of the stabilities of untested sequences. The disclosure shows that a chimera's thermostability can be predicted from the additive contributions of sequence fragments. Those contributions can be determined either by linear regression of stability-sequence data or, with less accuracy, from the frequencies with which the specific sequence fragments appear in folded vs. unfolded chimera population. Using these observations as the basis for predicting highly stable sequences, a diverse family of 40 thermostable cytochrome P450s whose half-lives of inactivation at 57° C. are as much as 100 times that of the most stable parent. Differing from any known natural P450 by up to 100 amino acid substitutions and from one another by as many as 88, the stable P450s are diverse, yet still retain catalytic activity. Some are significantly more active than the parent enzymes towards a nonnatural substrate, 2-phenoxyethanol. This stabilized protein family provides a unique ensemble for biotechnological applications and for studying sequence-stability-function relationships.

### BRIEF DESCRIPTION OF THE FIGURES

FIG. 1 shows thermostabilities of parental and chimeric cytochromes P450. The distribution of T₅₀ values for 185 chimeric cytochromes P450 has an average of 50.4° C. and standard deviation of 4.5° C. Thermostabilities for parents A1, A2 and A3 are indicated (solid lines), with four experimental replicate measurements for A2 to examine measurement variability (dotted lines, standard deviation of 1.0° C.)

FIG. 2A-B shows sequence elements contribute additively to thermostabilities of chimeric cytochromes P450. a, Predicted T₅₀ from a simple linear model correlates with the measured T₅₀ for 185 P450 chimeras with r=0.857. b, Linear model derived from data in a accurately predicts stabilities of 20 additional chimeras, including the most-stable P450 (MTP) (top rightmost point).

FIG. 3A-B depicts relative frequencies of sequence elements among folded chimeras correlates with relative stability contributions. a, Thermostability contributions of fragments from parents A1 and A3 relative to those from parent A2, obtained by linear regression analysis of 205 folded chimeras with measured T₅₀. b, Frequencies of fragments from parents A1 and A3 relative to those from parent A2 among folded chimeras. c, Relative fragment thermostability contributions correlate with their relative frequencies among folded chimeras.

FIG. 4A-D shows chimera thermostabilities and folding status predicted from sequence element frequencies in multiple sequence alignments of folded and unfolded proteins. a, Consensus energies computed from Boltzmann statistics and fragment frequencies of folded chimeras correlate with measured thermostabilities (T₅₀s). b, The distribution of consensus energies of 620 folded chimeras and 335 unfolded chimeras. Folded chimeras (dark grey) have lower consensus energies than unfolded chimeras (light grey). Overlap region is shown. The consensus energies were calculated as in a. Distributions are histogram densities. c, Consensus energies computed from Boltzmann statistics and fragment frequencies using folded and unfolded chimeras correlate with measured thermostabilities (T₅₀). d, Folded chimeras (dark grey) have lower consensus energies than unfolded chimeras (light grey). Overlap region is shown. Consensus energies were calculated as in c.

FIG. 5A-D shows linear regression analysis of protein stability. a. Predicted T₅₀ compared to experimental T₅₀for the training data set. The r value for the regression line is 0.901. Squares represent outlier points removed after training. b. Predicted T₅₀compared to measured T₅₀for the test data set. The r value for the regression line is 0.856. c. Prediction accuracy (indicated by correlation coefficient between predicted T₅₀and measured T₅₀) depends on the number of chimeras used for regression analysis. d. Prediction of T₅₀s of 6,561 members of the synthetic protein library.

FIG. 6 shows prediction accuracy (indicated by the Spearman rank-order correlation coefficient between predicted consensus energies and measured T₅₀) is related to the number of chimeras used for consensus analysis.

FIG. 7A-B shows sequence diversity for 40 stable chimeric cytochrome P450 heme domains and the three parent sequences. a. The number of amino acid differences between each pair of chimeras (black) and for parent-chimera pairs (grey). Pairwise sequence differences range from 7 to 167 amino acids. b. It is not possible to create a two-dimensional illustration with all chimera-chimera Euclidean distances perfectly proportional to the underlying sequence differences. A multi-dimensional scaling (XGOBI) was used to optimize a two-dimensional representation that minimizes the discrepancy between the Euclidean distances and the sequence differences.

FIG. 8 shows a comparison of the ranking performance using regression (circles) to the ranking performance using consensus (filled circles). The points represent the performance of each ranking method when partitioning the set of three parents and 205 chimeras with measured T₅₀ values into the top 10, 20, 30 . . . 200. For example, the y-positions of the leftmost points indicate that the consensus method correctly flags 4 of the top 10 chimeras while the regression method correctly flags 6. The x-positions of the leftmost points indicate that the consensus method correctly flags 96 of the bottom 99 chimeras while the regression method correctly flags 97. The regression model has superior ranking performance for all threshold choices.

FIG. 9 depicts the sequence domains.

FIG. 10 shows the amino acid sequence for CYP102A1.

FIG. 11 shows the amino acid sequence for CYP102A2.

FIG. 12 shows the amino acid sequence for CYP102A3.

FIG. 13A-B show an alignment of SEQ ID NOs:1-3.

### DETAILED DESCRIPTION

As used herein and in the appended claims, the singular forms “a,” “and,” and “the” include plural referents unless the context clearly dictates otherwise. Thus, for example, reference to “a domain” includes a plurality of such domains and reference to “the protein” includes reference to one or more proteins, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

Proteins fold into native structures determined by their amino acid sequences and thereby become biologically active. Stability of the native structure therefore plays a vital role in function, and also in protein turnover, genetic diseases, mutational tolerance, functional evolvability, and even the rate of evolution. Proteins with enhanced stability are of significant benefit in industrial applications, where they are better suited to formulation, long-term storage, and extended use in non-natural environments such as elevated temperature. Stabilized proteins are also better starting points for engineering, because their enhanced robustness to mutations makes it easier for them to acquire new functional properties.

The versatile cytochrome P450 family of heme-containing redox enzymes hydroxylate a wide range of substrates to generate products of significant medical and industrial importance. A particularly well-studied member of this diverse enzyme family, cytochrome P450 BM3 (CYP102A1, or “A1”; SEQ ID NO:1; see also GenBank Accession No. J04832, which is incorporated herein by reference) from Bacillus megaterium, has been engineered extensively for biotechnological applications that include fine chemical synthesis and producing human metabolites of drugs.

The disclosure demonstrates a new approach to making highly stable, functional proteins with diverse sequences by predicting the stable chimeras in a site-directed SCHEMA recombination library. Two approaches to analyzing a subset of the SCHEMA chimeras were shown to be effective. Both approaches assume that the fragments contribute in an additive manner to the overall stability, but estimate those contributions using different data. One calculates the contributions by linear regression of sequence-stability data, and the other is based on consensus analysis of the MSAs of folded versus unfolded proteins. Both approaches identify highly stable sequences; SCHEMA recombination ensures that they also retain biological function and exhibit high sequence diversity by conserving important functional residues while exchanging tolerant ones.

That fragments of the primary sequence contribute additively to stability may appear surprising, considering the cooperative nature of protein folding and many tertiary contacts in the native structure. The high degree of additivity observed in this study may be a feature of SCHEMA library design. SCHEMA identifies those sequence fragments that minimize the number of contacts, or interactions, that can be broken upon recombination. Two residues in a chimera are defined to have a contact if any heavy atoms are within 4.5 Å; the contact is broken if they do not appear together in any parent at the same positions. Among a total of about 500 contacts for a P450 chimera, an average of fewer than 30 were broken for the sequences in the SCHEMA library. The fragments that were swapped in this library have a high number of internal contacts; the inter-fragment contacts are either few or are conserved among the parents. Therefore, the fragments function as pseudo-independent structural modules that make roughly additive contributions to stability. The additivity was strong enough to enable detection of sequencing errors based on deviations from additivity, prediction of thermostabilities for uncharacterized chimeras with high accuracy, and prediction of the T₅₀ of the most stable chimera to within measurement error. This additivity enabled a new approach to stabilizing an entire protein family that does not require high throughput selection or screening.

An “amino acid” is a molecule having the structure wherein a central carbon atom (the -carbon atom) is linked to a hydrogen atom, a carboxylic acid group (the carbon atom of which is referred to herein as a “carboxyl carbon atom”), an amino group (the nitrogen atom of which is referred to herein as an “amino nitrogen atom”), and a side chain group, R. When incorporated into a peptide, polypeptide, or protein, an amino acid loses one or more atoms of its amino acid carboxylic groups in the dehydration reaction that links one amino acid to another. As a result, when incorporated into a protein, an amino acid is referred to as an “amino acid residue.”

“Protein” or “polypeptide” refers to any polymer of two or more individual amino acids (whether or not naturally occurring) linked via a peptide bond, and occurs when the carboxyl carbon atom of the carboxylic acid group bonded to the -carbon of one amino acid (or amino acid residue) becomes covalently bound to the amino nitrogen atom of amino group bonded to the -carbon of an adjacent amino acid. The term “protein” is understood to include the terms “polypeptide” and “peptide” (which, at times may be used interchangeably herein) within its meaning. In addition, proteins comprising multiple polypeptide subunits (e.g., DNA polymerase III, RNA polymerase II) or other components (for example, an RNA molecule, as occurs in telomerase) will also be understood to be included within the meaning of “protein” as used herein. Similarly, fragments of proteins and polypeptides are also within the scope of the invention and may be referred to herein as “proteins.” In one aspect of the disclosure, a stabilized protein comprises a chimera of two or more parental peptide segments.

A “peptide segment” refers to a portion or fragment of a larger polypeptide or protein. A peptide segment need not on its own have functional activity, although in some instances, a peptide segment may correspond to a domain of a polypeptide wherein the domain has its own biological activity. A stability-associated peptide segment is a peptide segment found in a polypeptide that promotes stability, function, or folding compared to a related polypeptide lacking the peptide segment. A destabilizing-associated peptide segment is a peptide segment that is identified as causing a loss of stability, function or folding when present in a polypeptide.

A particular amino acid sequence of a given protein (i.e., the polypeptide's “primary structure,” when written from the amino-terminus to carboxy-terminus) is determined by the nucleotide sequence of the coding portion of a mRNA, which is in turn specified by genetic information, typically genomic DNA (including organelle DNA, e.g., mitochondrial or chloroplast DNA). Thus, determining the sequence of a gene assists in predicting the primary sequence of a corresponding polypeptide and more particular the role or activity of the polypeptide or proteins encoded by that gene or polynucleotide sequence.

“Polynucleotide” or “nucleic acid sequence” refers to a polymeric form of nucleotides. In some instances a polynucleotide refers to a sequence that is not immediately contiguous with either of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides of the invention can be ribonucleotides, deoxyribonucleotides, or modified forms of either nucleotide. A polynucleotides as used herein refers to, among others, single-and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions.

In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term polynucleotide encompasses genomic DNA or RNA (depending upon the organism, i.e., RNA genome of viruses), as well as mRNA encoded by the genomic DNA, and cDNA. Polynucleotides encoding P450 from Bacillus megaterium see e.g., GenBank accession no. J04832 and subtilis are known.

A “nucleic acid segment,” “oligonucleotide segment” or “polynucleotide segment” refers to a portion of a larger polynucleotide molecule. The polynucleotide segment need not correspond to an encoded functional domain of a protein; however, in some instances the segment will encode a functional domain of a protein. A polynucleotide segment can be about 6 nucleotides or more in length (e.g., 6-20, 20-50, 50-100, 100-200, 200-300, 300-400 or more nucleotides in length). A stability-associated peptide segment can be encoded by a stability-associated polynucleotide segment, wherein the peptide segment promotes stability, function, or folding compared to a polypeptide lacking the peptide segment.

A chimera is a combination of at least two segments of at least two different parent proteins. As appreciated by one of skill in the art, the segments need not actually come from each of the parents, as it is the particular sequence that is relevant, and not the physical nucleic acids themselves. For example, a chimeric P450 will have at least two segments from two different parent P450s. The two segments are connected so as to result in a new P450. In other words, a protein will not be a chimera if it has the identical sequence of either one of the parents. A chimeric protein can comprise more than two segments from two different parent proteins. For example, there may be 2, 3, 4, 5-10, 10-20, or more parents for each final chimera or library of chimeras. The segment of each parent enzyme can be very short or very long, the segments can range in length of contiguous amino acids from 1 to the entire length of the protein. In one embodiment, the minimum length is 10 amino acids. In one embodiment, a single crossover point is defined for two parents. The crossover location defines where one parent's amino acid segment will stop and where the next parent's amino acid segment will start. Thus, a simple chimera would only have one crossover location where the segment before that crossover location would belong to one parent and the segment after that crossover location would belong to the second parent. In one embodiment, the chimera has more than one crossover location. For example, 2, 3, 4, 5, 6, 7, 8, 9, 10, 11-30, or more crossover locations. How these crossover locations are named and defined are both discussed below. In an embodiment where there are two crossover locations and two parents, there will be a first contiguous segment from a first parent, followed by a second contiguous segment from a second parent, followed by a third contiguous segment from the first parent. Contiguous is meant to denote that there is nothing of significance interrupting the segments. These contiguous segments are connected to form a contiguous amino acid sequence. For example, a P450 chimera from CYP102A1 (hereinafter “A1”) and CYP102A2 (hereinafter “A2”), with two crossovers at 100 and 150, could have the first 100 amino acids from A1, followed by the next 50 from A2, followed by the remainder of the amino acids from A1, all connected in one contiguous amino acid chain. Alternatively, the P450 chimera could have the first 100 amino acids from A2, the next 50 from A1 and the remainder followed by A2. As appreciated by one of skill in the art, variants of chimeras exist as well as the exact sequences. Thus, not 100% of each segment need be present in the final chimera if it is a variant chimera. The amount that may be altered, either through additional residues or removal or alteration of residues will be defined as the term variant is defined. Of course, as understood by one of skill in the art, the above discussion applies not only to amino acids but also nucleic acids which encode for the amino acids.

Protein stability is a key factor for industrial protein use (e.g., enzyme reaction) in denaturing conditions required for efficient product development and in therapeutic and diagnostic protein products. Methods for optimizing protein stability have included directed evolution and domain shuffling. However, screening and developing such recombinant libraries is difficult and time consuming.

Directed evolution has proven to be an effective technique for engineering proteins with desired properties. Because the probability of a protein retaining its fold and function decreases exponentially with the number of random substitutions introduced (Bloom et al., Proc. Natl Acad. Sci. USA, 102, 606-611, 2005), only a few mutations are made in each generation in order to maintain a reasonable fraction of functional proteins for screening (Voigt et al., Advances in Protein Chemistry, Vol 55, Academic Press, pp. 79-160, 2001). Creating libraries with higher levels of mutation while maintaining structure and function requires identifying mutations that are less likely to disrupt the structure (Lutz and Patrick, Curr. Opin. Biotechnol., 15, 291-297, 2004). One strategy to accomplish this is homologous recombination: mutations introduced by recombination are less deleterious than random mutations because they are compatible with the backbone structure (Drummond et al., Proc. Natl Acad. Sci. USA, 102, 5280-5385, 2005). Random recombination of highly similar proteins often generates libraries with a high fraction of functional sequences; however, as more distantly related proteins are recombined, the fraction of chimeric proteins that fold correctly decreases.

Efforts have been made to identify consensus mutations that provide stabilizing effects. Consensus stabilization has been shown to be effective in some cases and to some degree, but not all consensus mutations are stabilizing (e.g., more than 40% of the consensus residues identified from multiple sequence alignment of naturally occurring β-lactamases are in fact destabilizing rather than stabilizing (Amin et al. Prot. Eng. Des. & Sel., 17(11):787-793, 2004)). These methods have two problems: first single mutations generally have small effects on stability and second not all mutations can be combined such that the stabilizing effects can be properly measured.

Thus, methods of protein development have focused on providing stabilized proteins by generating a large number of recombined proteins and assaying each recombined protein for activity. A method of identifying stabilizing mutations is a first step in removing or narrowing possible candidates. For this reason it is of value to be able to make multiple versions of a protein that are stabilized. If one has many stable variants to choose from, then those variants that exhibit all of the properties of interest can be identified by appropriate analysis of those properties. The disclosure provides a method for making many (e.g., from 1 to many thousand) variants of a protein having amino acid sequences that may differ at multiple amino acid positions and that are stabilized and thus are likely to be functional. Such techniques for generating libraries of stabilized proteins have not previously been provided in the art.

A number of techniques are used for generating novel proteins including, for example, rational design, which uses computational methods to identify sites for introducing disulfide bonds; directed evolution; and consensus stabilization. The foregoing methods do not utilize a linear regression or consensus analysis to assist selectively designing stabilized proteins.

Recombination has been widely applied to accelerate in vitro protein evolution. In this process, the genetic information of several genes is exchanged to produce a library of recombined, recombinant mutants. These mutants are screened for improvement in properties of interest, such as stability, activity, or altered substrate specificity. In vitro recombination methods include DNA shuffling, random-priming recombination, and the staggered extension process (StEP). In DNA shuffling, the parental DNA is enzymatically digested into fragments. The fragments can be reassembled into offspring genes. In the random-priming method, template DNA sequences are primed with random-sequence primers and then extended by DNA polymerase to create fragments. The template is removed and the fragments are reassembled into full-length genes, as in the final step of DNA shuffling. In each of these methods, the number of cut points can be increased by starting with smaller fragments or by limiting the extension reaction. StEP recombination differs from the first two methods because it does not use gene fragments. The template genes are primed and extended before denaturation and reannealing. As the fragments grow, they reanneal to new templates and thus combine information from multiple parents. This process is cycled hundreds of times until a full-length offspring gene is formed. The foregoing methods are known in the art.

Recently, it has been shown that recombining genes that have evolved independently in nature is a powerful way to quickly accumulate large improvements in stability and function. Given the explosive growth in the gene databases due to the exhaustive sequencing of large numbers of organisms, the sequences of homologous genes are easily accessible. These sequences can be synthesized or cloned for evolution of protein functions by recombination methods described above and known in the art.

Common to these experimental approaches to recombination in vitro is that the genes are cut and reformed randomly, that is, there is little or no a priori input into the experimental protocol regarding which genes are chosen for recombination and where the cut points should occur, other than in regions of high sequence similarity. Using the SCHEMA method (described further herein) sequences are predicted that are more likely to generate diverse recombined, recombinant gene libraries and the desired improvements in the recombined, recombinant genes.

As a first step in performing any recombination techniques a set of related polypeptides is identified. The relatedness of the polypeptides can be determined in any number of ways known in the art. For example, polypeptides may be related structurally either in their primary sequence or in the secondary or tertiary sequence. Methods of identifying sequence identity or 3D structural similarities are known and are further described herein. Another method to identify a related polypeptide is through evolutionary analysis. Evolutionary trees have been developed for a large number of proteins and are available to those of skill in the art.

A parental sequence used as a basis for defining a set of related polypeptides can be provided by any of a number of mechanisms, including, but not limited to, sequencing, or querying a nucleic acid or protein database. Additionally, while the parental sequence can be provided in a physical sense (e.g., isolated or synthesized), typically the parental sequence or sequences are obtain in silico.

For embodiments of the disclosure involving amino acid sequences, the parental sequences typically are derived from a common family of proteins having similar three-dimensional structures (e.g., protein superfamilies). However, the nucleic acid sequences encoding these proteins might or might not share a high degree of sequence identity. As described later herein, the methods include assessing crossover positions using any number of techniques (e.g., SCHEMA etc.).

Sequence similarity/identity of various stringency and length can be detected and recognized using a number of methods or algorithms known to one of skill in the art. For example, many identity or similarity determination methods have been designed for comparative analysis of sequences of biopolymers, for spell-checking in word processing, and for data retrieval from various databases. With an understanding of double-helix pair-wise complement interactions among the four principal nucleobases in natural polynucleotides, models that simulate annealing of complementary homologous polynucleotide strings can also be used as a foundation of sequence alignment or other operations typically performed on the character strings corresponding to the sequences herein (e.g., word-processing manipulations, construction of figures comprising sequence or subsequence character strings, output tables, etc.). An example of a software package for calculating sequence identity is BLAST, which can be adapted to the disclosure by inputting character strings corresponding to the sequences herein.

After providing parental sequences, the sequences are aligned. In other embodiments, a plurality of parental sequences are provided, which are then aligned with either a reference sequence, or with one another. Alignment and comparison of relatively short amino acid sequences (for example, less than about 30 residues) is typically straightforward. Comparison of longer sequences can require more sophisticated methods to achieve optimal alignment of two sequences.

Optimal alignment of sequences can be performed, for example, by a number of available algorithms, including, but not limited to, the “local homology” algorithm of Smith and Waterman (Adv. Appl. Math. 2:482, 1981), the “homology alignment” algorithm of Needleman and Wunsch (J. Mol. Biol. 48:443, 1970), the “search for similarity” method of Pearson and Lipman (Proc. Natl. Acad. Sci. USA 85:2444, 1988), or by computerized implementations of these algorithms (e.g., GAP, BESTFIT, FASTA and TFASTA available in the Wisconsin Genetics Software Package Release 7.0, Genetics Computer Group, 575 Science Dr., Madison, Wis.; and BLAST, see, e.g., Altschul et al., Nuc. Acids Res. 25:3389-3402, 1977 and Altschul et al., J. Mol. Biol. 215:403-410, 1990). Alternatively, the sequences can be aligned by inspection. Generally the best alignment (i.e., the relative positioning resulting in the highest percentage of sequence identity over the comparison window) generated by the various methods is selected. However, in certain embodiments of the disclosure, the best alignment may alternatively be a superpositioning of selected structural features, and not necessarily the highest sequence identity.

The term “sequence identity” means that two amino acid sequences are substantially identical (i.e., on an amino acid-by-amino acid basis) over a window of comparison. The term “sequence similarity” refers to similar amino acids that share the same biophysical characteristics. The term “percentage of sequence identity” or “percentage of sequence similarity” is calculated by comparing two optimally aligned sequences over the window of comparison, determining the number of positions at which the identical residues (or similar residues) occur in both polypeptide sequences to yield the number of matched positions, dividing the number of matched positions by the total number of positions in the window of comparison (i.e., the window size), and multiplying the result by 100 to yield the percentage of sequence identity (or percentage of sequence similarity). With regard to polynucleotide sequences, the terms sequence identity and sequence similarity have comparable meaning as described for protein sequences, with the term “percentage of sequence identity” indicating that two polynucleotide sequences are identical (on a nucleotide-by-nucleotide basis) over a window of comparison. As such, a percentage of polynucleotide sequence identity (or percentage of polynucleotide sequence similarity, e.g., for silent substitutions or other substitutions, based upon the analysis algorithm) also can be calculated. Maximum correspondence can be determined by using one of the sequence algorithms described herein (or other algorithms available to those of ordinary skill in the art) or by visual inspection.

As applied to polypeptides, the term substantial identity or substantial similarity means that two peptide sequences, when optimally aligned, such as by the programs BLAST, GAP or BESTFIT using default gap weights or by visual inspection, share sequence identity or sequence similarity. Similarly, as applied in the context of two nucleic acids, the term substantial identity or substantial similarity means that the two nucleic acid sequences, when optimally aligned, such as by the programs BLAST, GAP or BESTFIT using default gap weights (described in detail below) or by visual inspection, share sequence identity or sequence similarity.

One example of an algorithm that is suitable for determining percent sequence identity or sequence similarity is the FASTA algorithm, which is described in Pearson, W. R. & Lipman, D. J., (1988) Proc. Natl. Acad. Sci. USA 85:2444. See also, W. R. Pearson, (1996) Methods Enzymology 266:227-258. Preferred parameters used in a FASTA alignment of DNA sequences to calculate percent identity or percent similarity are optimized, BL50 Matrix 15: −5, k-tuple=2; joining penalty=40, optimization=28; gap penalty −12, gap length penalty=−2; and width=16.

Another example of a useful algorithm is PILEUP. PILEUP creates a multiple sequence alignment from a group of related sequences using progressive, pairwise alignments to show relationship and percent sequence identity or percent sequence similarity. It also plots a tree or dendogram showing the clustering relationships used to create the alignment. PILEUP uses a simplification of the progressive alignment method of Feng & Doolittle, (1987) J. Mol. Evol. 35:351-360. The method used is similar to the method described by Higgins & Sharp, CABIOS 5:151-153, 1989. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity (or percent sequence similarity) relationship using the following parameters: default gap weight (3.00), default gap length weight (0.10), and weighted end gaps. PILEUP can be obtained from the GCG sequence analysis software package, e.g., version 7.0 (Devereaux et al., (1984) Nuc. Acids Res. 12:387-395).

Another example of an algorithm that is suitable for multiple DNA and amino acid sequence alignments is the CLUSTALW program (Thompson, J. D. et al., (1994) Nuc. Acids Res. 22:4673-4680). CLUSTALW performs multiple pairwise comparisons between groups of sequences and assembles them into a multiple alignment based on sequence identity. Gap open and Gap extension penalties were 10 and 0.05 respectively. For amino acid alignments, the BLOSUM algorithm can be used as a protein weight matrix (Henikoff and Henikoff, (1992) Proc. Natl. Acad. Sci. USA 89:10915-10919).

Another method of determining relatedness is through protein and polynucleotide alignments. Common methods include using sequence based searches available on-line and through various software distribution routes. Homology or identity at the amino acid or nucleotide level can be determined by BLAST (Basic Local Alignment Search Tool) and by ClustalW analysis using the algorithm employed by the programs blastp, blastn, blastx, tblastn and tblastx (Karlin et al., Proc. Natl. Acad. Sci. USA 87, 2264-2268, 1990; Thompson et al., Nucleic Acids Res 22,4673-4680, 1994; and Altschul, J. Mol. Evol. 36, 290-300, 1993, (fully incorporated by reference) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases (see Altschul et al., Nature Genetics 6, 119-129, 1994, which is fully incorporated by reference). The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., Proc. Natl. Acad. Sci. USA 89, 10915-10919, 1992, fully incorporated by reference). For blastn, the scoring matrix is set by the ratios of M (i.e., the reward score for a pair of matching residues) to N (i.e., the penalty score for mismatching residues), wherein the default values for M and N are 5 and −4, respectively.

Accordingly, by using such methods families or groups of structurally related polypeptides can be identified. Typically the protein homology (whether they are evolutionarily, and therefore structurally, related) is determined primarily by sequence similarity (sequences are more similar than expected at random). Sequences that are as low as 15-20% similar by alignments are likely related and encode proteins with similar structures. Additional structural relatedness can be determine using any number of further techniques including, but not limited to, X-ray crystallography, NMR, searching a protein structure databases, homology modeling, de novo protein folding, and computational protein structure prediction. Such additional techniques can be used alone or in addition to sequence-based alignment techniques. In one aspect, the degree of similarity/identity between two proteins or polynucleotide sequences should be at least about 20% or more (e.g., 30%, 35%, 40%, 45%, 50%, 55%, 60%, 65%, 70%, 75%, 80%, 85%, 90%, 95%, 98% or 99%).

In some aspects, parent sequences are chosen from a database of sequences, by a sequence homology search such as BLAST. Parental sequences will typically be between about 20% and 95% identical, typically between 35 and 80% identical. The lower the identity, the more the mutation level (and possibly the greater the possible stability enhancement and functional variation in the resulting sequences) following recombination between parental strands. The higher the identity, the higher the probability the sequences will fold and function.

Thermodynamic stability is an important biological property that has evolved to an optimal level to fit the functional needs of proteins. Therefore, investigating the stability of proteins is important not only because it affords information about the physical chemistry of folding, but also because it can provide important biological insights. A proper understanding of protein stability is also useful for technological purposes. The ability to rationally make proteins of high stability, low aggregation or low degradation rates will be valuable for a number of applications. For example, proteins that can resist unfolding can be used in industrial processes that require enzyme catalysis at high temperatures (Van den. Burg et al., Proc. Natl. Acad. Sci. U.S.A. 95(5): 2056-60, 1998); and the ability to produce proteins with low degradation rates within the cell can help to maximize production of recombinant proteins (Kwon et al., Protein Eng. 9(12): 1197-202, 1996).

Stability measurements can also be used as probes of other biological phenomena. The most basic of these phenomena is biological activity. The ability of proteins to populate their native states is a universal requirement for function. Therefore, stability can be used as a convenient, first level assay for function. For example, libraries of polypeptide sequences can be tested for stability in order to select for sequences that fold into stable conformations and might potentially be active (Sandberg et al., Biochem. 34: 11970-78, 1995). Heme domains of cytochromes can be assayed for proper folding using CO-binding to the iron/heme. The heme domain for SEQ ID NO:1 extends from about amino acid residue 1 to about 434; and for SEQ ID NO:2 or 3 extends from about amino acid residue 1 to about 436.

Changes in stability can also be used to detect binding. When a ligand binds to the native conformation of a protein, the global stability of a protein is increased (Schellman, Biopolymers 14: 999-1018, 1975; Pace & McGrath, (1980) J. Biol. Chem. 255: 3862-65; Pace & Grimsley, Biochem. 27: 3242-46, 1988). The binding constant can be measured by analyzing the extent of the stability increase. This strategy has been used to analyze the binding of ions and small molecules to a number of proteins (Pace & McGrath, (1980) J. Biol. Chem. 255: 3862-65; Pace & Grimsley, (1988) Biochem. 27: 3242-46; Schwartz, (1988) Biochem. 27: 8429-36; Brandts & Lin, (1990) Biochem. 29: 6927-40; Straume & Freire, (1992) Anal. Biochem. 203: 259-68; Graziano et al., (1996) Biochem. 35: 13386-92; Kanaya et al., (1996) J. Biol. Chem. 271: 32729-36).

The linkage between stability and binding has recently been implemented as a method to detect ligand binding (U.S. Pat. No. 5,679,582 to Bowie & Pakula). This method, however, does not take advantage of the high sensitivity available from an analytical technique such as MALDI mass spectrometry, and cannot be employed at the low protein levels that MALDI mass spectrometry can detect. Moreover, proteolytic methods can require additional steps to isolate and analyze proteolytic fragments and cannot be performed in an in vivo setting. Finally, this method cannot be employed to generate quantitative measurements of protein stability.

The expressed chimeric recombinant proteins are measured for stability and/or biological activity. Techniques for measuring stability and activity are known in the art and include, for example, the ability to retain function (e.g. enzymatic activity) at elevated temperature or under ‘harsh’ conditions of pH, salt, organic solvent, and the like; and/or the ability to maintain function for a longer period of time (e.g., in storage in normal conditions, or in harsh conditions). Function will of course depend upon the type of protein being generated and will be based upon its intended purpose. For example, P450 mutants can be tested for the ability to convert alkanes to alcohols under various conditions of pH, solvents and temperature.

The best methodology for protein stabilization depends on the target protein and the relative ease with which folding status and stability are measured. The linear regression model uses stability data, which are often more difficult to obtain than a simple determination of folding status. The linear regression model, however, requires fewer measurements and always predicted more true positives with fewer false positives than the consensus approach based on folding status (FIG. 8). While the linear regression model predicted absolute thermostabilities with higher accuracy than the consensus model, the latter nonetheless reliably predicted highly stable chimeras. The two approaches have significant overlaps in their predicted stable sequences, including the MTP. Eight of the top seventeen stable chimeras predicted by consensus have predicted T₅₀>60° C. by linear regression (Table 3). Among the 50 chimeras with lowest consensus energies, 28 sequences are also predicted very stable by linear regression, and all 17 chimeras constructed have measured T₅₀>58° C. (there are 87 chimeras with predicted T₅₀>58° C.). Nineteen of the top 50 stable chimeras predicted by linear regression have consensus energies ranked in the top 50. The consensus analysis can also be performed using multiple sequence alignments that are based on functional status (functional or not) rather than folding status, since most chimeras that fold also retain functions shared by the parent proteins.

Consensus stabilization is based on the idea that the frequencies of sequence elements correlate with their corresponding stability contributions. This correlation is typically assumed to follow a Boltzmann-like exponential relationship. Such a relationship is most sensible if, in analogy to statistical mechanics, the sequences are randomly sampled from the ensemble of all folded sequences. Natural sequences are related by divergent evolution and may not comprise such a sample. The chimeric data set, in contrast, represents a large and nearly random sample of all the 6,561 possible chimeras. Dramatic support for the fundamental assumptions underlying consensus stabilization approaches were found: sequence elements contribute additively to stability, stabilizing fragments occur at higher frequencies among folded sequences, and the consensus sequence is the most stable in the ensemble. Taking advantage of the unique access to unfolded sequences, failure to fold is also informative: incorporation of sequence element frequencies among unfolded sequences significantly improves predictions of both relative stability and protein folding status. These results demonstrate the tolerance of the consensus stabilization idea to different ensembles (chimeric libraries versus evolved families) and sequence changes (recombination versus stepwise mutation), supporting its use as a reliable method for protein stabilization.

The ensemble of 40 stable, homologous P450s also represents a valuable resource for studying the relationship between enzyme stability and activity. Comparative studies of homologous enzymes from mesophilic and thermophilic organisms often find that the highly stable enzymes from thermophiles are less active at room temperature than their marginally stable mesophilic counterparts. These findings have been used to argue that highly stable proteins are inherently less capable of functioning as active enzymes at room temperature, perhaps due to decreased flexibility. An alternative viewpoint holds that proteins from thermophiles are poor enzymes at low-temperature (e.g. room temperature) because they have evolved under pressure to function at high temperatures, just as proteins from mesophiles are marginally stable because they have never been selected to fold at higher temperatures. This viewpoint that the poor low-temperature activity of naturally thermostable enzymes represents evolutionary statistics rather than an inherent biophysical tradeoff has anecdotal support from engineering experiments that have dramatically stabilized proteins while retaining their room-temperature activity. The current data, in which a large set of proteins with varying stabilities has been generated by recombination without evolutionary selection for either activity or stability, provide a more rigorous test. Over half of the 40 thermostable chimeras in Table 3 are also more active on 2-phenoxyethanol than the most active parent, demonstrating that there is no fundamental biophysical tradeoff between stability and activity on this substrate. Such trade-offs, if they exist, must be connected to significantly more optimized functions.

The disclosure demonstrates that chimeric proteins exhibit a broad range of stabilities, and that stability of a given folded sequence can be predicted based on data (either stability or folding status) from a limited sampling of the chimeric library. Using this information, dozens of diverse, highly stable proteins were created.

The following examples are meant to further explain, but not limited the foregoing disclosure or the appended claims.

### EXAMPLES

Thermostability measurements. Cell extracts were prepared and P450 concentrations were determined as reported previously¹³. Cell extract samples containing 4 μM of P450 were heated in a thermocycler over a range of temperatures for 10 minutes followed by rapid cooling to 4° C. for 1 minute. The precipitate was removed by centrifugation. The P450 remaining in the supernatant was measured by CO-difference spectroscopy. T₅₀, the temperature at which 50 percent of protein irreversibly denatured after a 10-min incubation, was determined by fitting the data to a two-state denaturation model. To check the variability and reproducibility of the measurement, four parallel independent experiments (from cell culture to T₅₀ measurement) were conducted on A2, which yielded an average T₅₀ of 43.6° C. and a standard deviation (σ_{M}) of 1.0° C. For some sequences, T₅₀ s were measured twice, and the average of all the measurements was used in the analysis.

Linear regression. The linear model

$${T}_{50} = {a}_{0} + {\sum }_{i }^{ } {\sum }_{j }^{ } {a}_{ij} {x}_{ij}$$

was used for regression, where T₅₀ is the dependent variable and fragments xᵢⱼ (from the i^{th} position and j^{th} parent, where i=1, 2, . . . 8 and j=1 or 3) are the independent variables. The were dummy-coded, such that if a chimera took fragment 1 from parent 1, x₁₁=1 and x₁₃=0. Parent A2 was used as the reference for all eight fragments, so the constant term (a₀) is the predicted T₅₀ of A2. The thermostability contribution of each fragment relative to the corresponding A2 fragment is given by the regression coefficient aᵢⱼ. Regression was performed using SPSS (SPSS for Windows, Rel. 11.0.1. 2001. Chicago: SPSS Inc.).

Construction of thermostable chimeric cytochrome P450s. To construct a given stable chimera, two chimeras having parts of the targeted gene (e.g. 21311212 and 11312333 for the target chimera 21312333) were selected as templates. The target gene was constructed by overlap extension PCR, cloned into the pCWori expression vector, and transformed into the catalase-free E. coli strain SN0037. All constructs were confirmed by sequencing.

Enzyme activity assay. Activity on 2-phenoxyethanol was analyzed in 96-well plates using the 4-aminoantipyrine (4-AAP) assay. 80 μl of P450 chimera (4 μM) was mixed 20 μl of 2-phenoxyethanol (3 M) in each well. The reaction was initiated by adding 20 μl of 120 mM hydrogen peroxide. The reaction mixture was incubated at room temperature for two hours. Then 50 μl of basic buffer (0.2 M NaOH and 4 M Urea) was added into the reaction mixture to raise the pH for the 4-AAP assay. 25 μl of 0.6% 4-AAP was added, the reading at 500 nm was taken for zeroing, and then 25 μl of 0.6% potassium persulfate was added. After incubation of 10 minutes at room temperature, the absorbance at 500 nm was recorded. The total turnover number (TTN) was calculated and then normalized to the most active parent, A1.

To generate a library of novel CYP102A sequences for these applications, a structure-guided SCHEMA recombination of the heme domains of CYP102A1 and its homologs CYP102A2 (A2) and CYP102A3 (A3) was used to create at least 2,300 new, properly folded and catalytically active enzymes. The folded chimeras exhibit a great deal of sequence diversity, differing from the closest parent sequence by an average of 72 amino acid substitutions. Some of these chimeric P450s were shown to be more stable than any of the parents.

The SCHEMA library was constructed by site-directed recombination at seven crossover sites, so that a chimeric P450 sequence is made up of eight fragments, each chosen from one of the three parents. The thermostabilities of a subset of the folded chimeras were measured and analyzed the relationship between sequence and stability. Thermostability is well described by a model that assumes the contributions of the chimera's eight sequence fragments are additive. The sequences of 620 folded and 335 unfolded chimeras were examined and found that the most thermostable chimeras tend to contain ‘consensus fragments’, or those appearing more frequently among the folded chimeras and less frequently in the unfolded ones. Chimera thermostability can thus be predicted by determining either the folding status or thermostabilities of a small sampling of the library. Based on these results, chimeras were predicted, constructed and characterized; 40 chimeric cytochrome P450s that are highly stable, catalytically active and have sequences that are significantly different from any known P450.

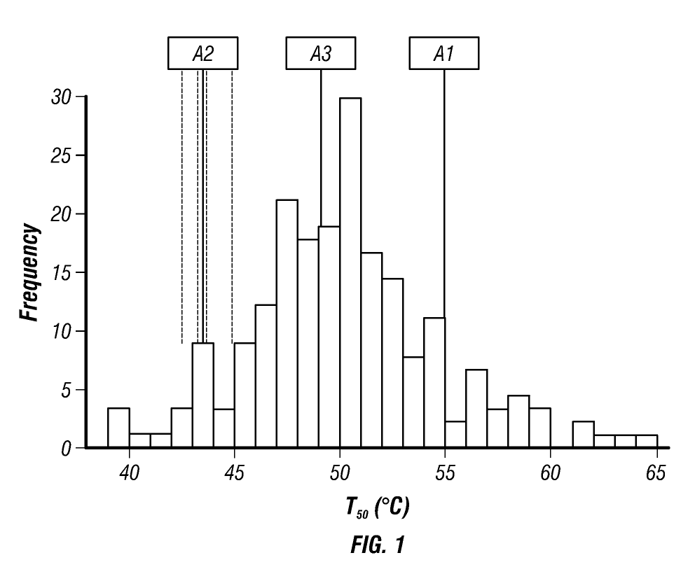

Thermostabilities of chimeric P450 heme domains. 620 folded and 335 unfolded chimeric P450 heme domains from a SCHEMA library constructed from three parents (CYP102A1, A2 and A3 (SEQ ID NO:1-3) have been provided. The 3⁸=6,561 possible chimeric sequences are written according to fragment composition: 23121321, for example, represents a protein which inherits the first fragment from parent A2, the second from A3, the third from A1, and so on. To determine the relationship between sequence and stability, thermostabilities of 185 folded P450 chimeras were measured (Table 1) in the form of T₅₀, the temperature at which 50% of the protein irreversibly denatured after incubation for ten minutes. The parental proteins have T₅₀ values of 54.9° C. (A1), 43.6° C. (A2) and 49.1° C. (A3). The T₅₀ distribution of the chimeras, shown in FIG. 1, has an average of 50.3° C. and a standard deviation of 4.5° C. This subset of the folded P450s contains many that are more stable than the most stable parent (A1).

TABLE 1
T₅₀ values and sequences of 205 chimeric cytochromes P450.

| Sequence | T₅₀ (° C.) |
|---|---|
| 32233232 | 39.8 |
| 32313233 | 52.9 |
| 21133233 | 48.8 |
| 31312113 | 45.0 |
| 21332223 | 48.3 |
| 21312323 | 61.5 |
| 22312322 | 54.6 |
| 21212112 | 51.2 |
| 23133121 | 47.3 |
| 11312233 | 51.6 |
| 21133312 | 45.4 |
| 21133313 | 50.8 |
| 11332233 | 43.3 |
| 31212332 | 53.4 |
| 12211232 | 49.1 |
| 31312133 | 52.6 |
| 12232332 | 39.2 |
| 22133232 | 47.9 |
| 22233221 | 46.8 |
| 23113323 | 51.0 |
| 11332212 | 47.8 |
| 32332231 | 49.4 |
| 22132331 | 53.3 |
| 23313111 | 56.9 |
| 23112323 | 46.0 |
| 11113311 | 51.2 |
| 21232233 | 50.6 |
| 12332233 | 47.1 |
| 23333311 | 45.7 |
| 32132233 | 42.9 |
| 22331123 | 47.9 |
| 12212332 | 48.4 |
| 31212323 | 48.7 |
| 21132323 | 50.1 |
| 23332231 | 51.4 |
| 12112333 | 50.9 |
| 22133212 | 47.2 |
| 31113131 | 54.9 |
| 23313333 | 61.2 |
| 21113133 | 51.9 |
| 21111323 | 54.4 |
| 22212123 | 47.7 |
| 12211333 | 50.6 |
| 23113112 | 46.3 |
| 21313122 | 50.5 |
| 23112333 | 54.3 |
| 12213212 | 44.0 |
| 23132233 | 43.6 |
| 21313311 | 56.9 |
| 21332231 | 60.0 |
| 23133233 | 43.1 |
| 32312322 | 49.1 |
| 32312231 | 52.6 |
| 21232332 | 49.3 |
| 31331331 | 47.3 |
| 21132222 | 45.6 |
| 21212333 | 63.2 |
| 21231233 | 50.6 |
| 22212322 | 50.7 |
| 21112122 | 50.3 |
| 22111223 | 51.3 |
| 23233212 | 39.5 |
| 31312212 | 48.9 |
| 32211323 | 46.6 |
| 21213231 | 54.9 |
| 21332312 | 52.9 |
| 22332211 | 53.0 |
| 22113323 | 53.8 |
| 22113332 | 48.7 |
| 22213132 | 52.0 |
| 31213332 | 50.8 |
| 22113211 | 51.1 |
| 22313323 | 60.0 |
| 32333233 | 47.2 |
| 22331223 | 51.7 |
| 23333233 | 51.0 |
| 22333332 | 49.0 |
| 23332331 | 48.0 |
| 21233132 | 42.4 |
| 13333211 | 45.7 |
| 22232331 | 50.5 |
| 22313233 | 58.5 |
| 31311233 | 56.9 |
| 21132321 | 49.3 |
| 12322333 | 47.9 |
| 23313233 | 56.3 |
| 21332322 | 48.8 |
| 22132231 | 53.0 |
| 21113312 | 53.0 |
| 22312223 | 56.2 |
| 23332223 | 46.7 |
| 32212323 | 48.4 |
| 21212111 | 57.2 |
| 31212212 | 47.1 |
| 22232121 | 49.7 |
| 21232212 | 47.8 |
| 21333223 | 49.1 |
| 23213232 | 48.5 |
| 22113232 | 51.1 |
| 11331333 | 46.3 |
| 22333321 | 49.2 |
| 21232321 | 46.0 |
| 32212231 | 47.4 |
| 23212212 | 48.0 |
| 22113223 | 49.9 |
| 22233211 | 46.3 |
| 23213311 | 49.5 |
| 31212321 | 44.9 |
| 23112233 | 51.0 |
| 32332323 | 48.5 |
| 22112223 | 52.8 |
| 32313231 | 52.5 |
| 32132232 | 42.5 |
| 22232233 | 49.6 |
| 22232322 | 45.4 |
| 22333211 | 50.7 |
| 22332223 | 52.4 |
| 23213212 | 49.0 |
| 23333213 | 50.1 |
| 31312233 | 57.9 |
| 22232333 | 53.7 |
| 31333233 | 46.5 |
| 22213212 | 50.5 |
| 22132212 | 46.6 |
| 21332233 | 58.9 |
| 23333131 | 50.5 |
| 31312332 | 54.9 |
| 21333221 | 51.3 |
| 22333223 | 49.9 |
| 21111333 | 62.4 |
| 12212212 | 44.8 |
| 11313233 | 48.3 |
| 32113232 | 47.9 |
| 21113322 | 50.4 |
| 31313232 | 51.9 |
| 31332233 | 49.9 |
| 21133232 | 46.4 |
| 22112211 | 54.7 |
| 21333333 | 58.0 |
| 22213223 | 50.8 |
| 21332112 | 50.4 |
| 21331332 | 52.0 |
| 11313333 | 53.8 |
| 32311323 | 52.0 |
| 23132231 | 48.0 |
| 12232232 | 40.9 |
| 21212231 | 59.9 |
| 21132212 | 48.8 |
| 23133311 | 44.2 |
| 22113111 | 49.2 |
| 23212211 | 50.7 |
| 21132112 | 47.1 |
| 23132311 | 44.5 |
| 23213333 | 56.1 |
| 21333233 | 54.2 |
| 22233212 | 44.0 |
| 21313112 | 54.8 |
| 31213233 | 50.6 |
| 22132113 | 40.6 |
| 31112333 | 55.7 |
| 31212331 | 51.8 |
| 22232222 | 47.5 |
| 23332221 | 46.4 |
| 21332131 | 58.5 |
| 23231233 | 45.5 |
| 22111332 | 50.9 |
| 23312121 | 49.3 |
| 22332222 | 50.3 |
| 23312323 | 53.8 |
| 21131121 | 53.0 |
| 32212232 | 48.8 |
| 22112323 | 55.3 |
| 21232232 | 49.5 |
| 11212333 | 50.4 |
| 31212232 | 51.0 |
| 23213211 | 47.4 |
| 11331312 | 43.5 |
| 23331233 | 50.9 |
| 22133323 | 49.4 |
| 33333233 | 46.3 |
| 22233323 | 48.4 |
| 32232131 | 43.9 |
| 31312323 | 52.3 |
| 21313313 | 64.4 |
| 22333231 | 53.1 |
| 22232123 | 43.1 |
| 33312333 | 54.7 |
| 22313232 | 58.8 |
| 22312111 | 53.0 |
| 32212233 | 49.9 |
| 21212321 | 53.3 |
| 21333211 | 55.9 |
| 22232212 | 46.2 |
| 23313323 | 50.9 |
| 32312333 | 57.8 |
| 12313331 | 51.2 |
| 21311331 | 62.9 |
| 21313231 | 61.0 |
| 22312133 | 57.1 |
| 22312231 | 60.0 |
| 22312311 | 55.6 |
| 22312332 | 59.1 |
| 22312333 | 63.5 |
| 21312333 | 64.4 |
| 21312123 | 60.8 |

Note:
The first 185 chimeras are those for data training and testing, and the last 20 chimeras (bold) are those used to test the linear regression model.

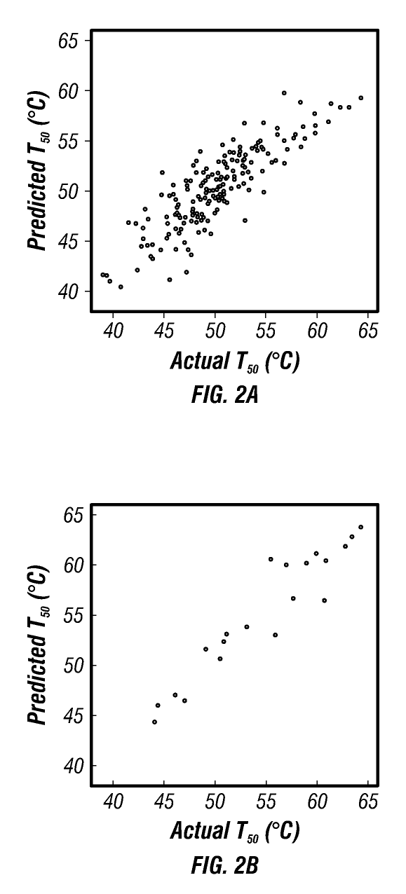

Linear regression analysis of chimera thermostability. The T₅₀ values of the 185 chimeric P450s were analyzed by linear regression, under the implicit assumption that each fragment contributes independently to protein thermostability. Regression of T₅₀ against chimera fragment composition revealed a strong linear correlation between predicted and observed T₅₀ over all 185 chimeras: Pearson r=0.857 (FIG. 2a) (Table 2).

TABLE 2
Thermostability contribution from each fragment
calculated by linear regression.

| | Relative thermostability contribution (° C.) | | |
|---|---|---|---|
| Regression | 185 chimeras | 140 chimeras | 205 chimeras |
| coefficient | (no training) | (with training) | (with training) |
| a₀ | 47.0 | 42.4 | 43.0 |
| a₁₁ | −7.2 | −7.4 | −7.6 |
| a₁₃ | −5.7 | −6.2 | −6.1 |
| a₂ₗ | 1.3 | 1.0 | 1.4 |
| a₂₃ | −3.1 | −4.3 | −3.9 |
| a₃₁ | 0.2 | 0.3 | −0.1 |
| a₃₃ | 3.9 | 4.8 | 4.2 |
| a₄₁ | 5.8 | 7.5 | 6.8 |
| a₄₃ | 0.0 | 1.7 | 0.9 |
| a₅₁ | −0.2 | −1.2 | −1.0 |
| a₅₃ | −0.9 | −1.7 | −1.4 |
| a₆₁ | −1.4 | −1.8 | −2.2 |
| a₆₃ | 1.2 | 1.0 | 1.1 |
| a₇₁ | 1.4 | 2.9 | 2.5 |
| a₇₃ | 3.5 | 4.5 | 4.2 |
| a₈₁ | 2.9 | 2.0 | 2.0 |
| a₈₃ | 2.4 | 0.6 | 3.0 |

Note:
The thermostability contribution of each fragment shown is relative to the corresponding fragment from parent A2, which was used as the reference.

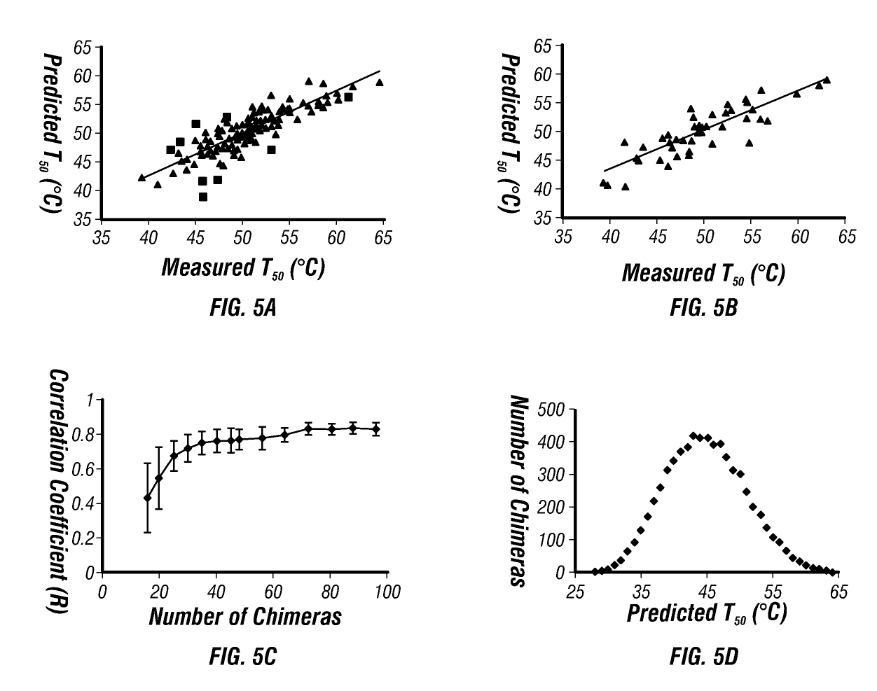

To examine whether the results allow generalization from one data subset to another and address the possibility of over-fitting, the data was randomly divided into two parts, a training set (140 data points) and a test set (45 data points). The standard deviation of regression (σ_{R}) and measurement (σ_{M}=1.0° C.) were used to guide the data training. After each training cycle, every data point was weighted in terms of its role in determining the regression line. If the prediction error (the temperature difference between the predicted T₅₀ and measured one) of a data point was more than 2σ_{R}, it was removed. When σ_{R} was less than 2σ_{M} (2.0° C.), the training process stopped. After two training cycles, a σ_{R} of 1.9° C. was achieved. After removing only 8 outliers, r for the training set was improved from 0.847 to 0.901 (FIG. 5a). When the trained regression parameters (Table 2) were used to predict thermostabilities of proteins in the test data set, r=0.856, indicating that additive contributions derived from one group of proteins can be used to accurately predict thermostabilities of another group (FIG. 5b). The linear regression model was further confirmed by 10-fold cross-validation.

The model parameters obtained from the training set (Table 2) were used to predict that the most thermostable P450 (MTP) chimera in the library would have a T₅₀ of 63.8° C. and fragment composition 21312333. This sequence was constructed, expressed and characterized; its T₅₀ of 64.4° C., within measurement error of the predicted value, made it 9.5° C. more stable than the most thermostable parent, A1. It was in fact the most stable of all 239 chimeras characterized to date. To further test the model predictions, the T₅₀ values of nineteen additional chimeras from the subset of 620 folded chimeras were measured, seven predicted to be highly thermostable and twelve picked at random (Table 1). Predicted and measured T₅₀ values for all 20 new P450s, including the MTP, correlated extremely well (r =0.949) (FIG. 2b).

In the absence of noise, determining the weights for predictor variables in a regression model only requires making as many measurements as there are predictor variables. In the presence of noise, additional measurements will tend to increase the accuracy of the predictions. A certain number of sequences from the 205 chimeras with measured T₅₀s were randomly selected and tested the ability of regression models based on these sequences to predict the T₅₀s of the remaining chimeras. 35 to 40 measurements were sufficient for accurate predictions of chimera stability, although slight improvements in prediction accuracy could be seen with more data points (FIG. 5c).

Protein stabilization by additivity of fragment contributions. Linear regression model parameters obtained from 205 T₅₀ measurements (Table 2) were used to predict T₅₀ values for all 6,561 chimeras in the SCHEMA P450 library (FIG. 5d). A significant number (˜300) of chimeras are predicted to be more stable than the most stable parent. Those with predicted T₅₀ values greater or equal to 60° C. (total of 31) were selected for construction and further characterization. Five were identified previously; the remaining 26 were constructed. All 31 predicted stable chimeras were stable, with T₅₀ between 58.5° C. and 64.4° C. (Table 3). The stability predictions were quite accurate, with root mean square deviations between the predicted and measured T₅₀ values of 1.5° C., close to the measurement error (1.0° C.)

TABLE 3
A stabilized cytochrome P450 heme domain family.

| | Predicted | Measured | | | Predicted | Measured | |
|---|---|---|---|---|---|---|---|
| Sequence | T₅₀ (C.) | T₅₀ (C.) | Activity³ | Sequence | T₅₀ (C.) | T₅₀ (C.) | Activity |
| 21312333^{1,2} | 63.8 | 64.4 | 1.0 | 21311231¹ | 60.7 | 63.2 | 0.8 |
| 21312331^{1,2} | 62.8 | 60.6 | 3.1 | 22312313¹ | 60.6 | 61.0 | 2.5 |
| 21311333¹ | 62.8 | 59.2 | 2.5 | 21313313¹ | 60.6 | 61.9 | 4.7 |
| 21312233^{1,2} | 62.7 | 63.1 | 0.6 | 22311331¹ | 60.4 | 58.9 | 5.1 |
| 22312333^{1,2} | 62.4 | 63.5 | 1.9 | 21312133¹ | 60.4 | 60.1 | 2.8 |
| 21313333^{1,2} | 62.4 | 62.9 | 3.8 | 22312231¹ | 60.3 | 61.4 | 2.3 |
| 21312313¹ | 62.0 | 62.2 | 2.8 | 21313231¹ | 60.3 | 61.0 | 1.8 |
| 21311331¹ | 61.8 | 62.9 | 1.0 | 22311233¹ | 60.3 | 60.9 | 3.1 |
| 21312231^{1,2} | 61.7 | 62.8 | 1.0 | 21311311¹ | 60.1 | 61.0 | 3.2 |
| 21311233¹ | 61.7 | 62.7 | 0.7 | 22313331¹ | 60.0 | 58.5 | 7.2 |
| 21313331¹ | 61.4 | 62.2 | 5.5 | 21312211¹ | 60.0 | 59.3 | 2.8 |
| 22312331¹ | 61.4 | 59.3 | 5.1 | 21212333² | 59.6 | 63.2 | 0.4 |
| 22311333¹ | 61.4 | 60.1 | 4.7 | 21112333² | 59.5 | 61.6 | 1.1 |
| 22312233^{1,2} | 61.3 | 61.0 | 2.7 | 21212233² | 58.5 | 60.0 | 1.3 |
| 21313233^{1,2} | 61.2 | 60.0 | 3.3 | 21112331² | 58.5 | 61.6 | 0.6 |
| 21312311¹ | 61.1 | 59.1 | 3.0 | 21112233² | 58.4 | 58.7 | 0.7 |
| 22313333¹ | 61.0 | 64.3 | 9.0 | 22212333² | 58.2 | 58.2 | 3.2 |
| 21311313¹ | 61.0 | 61.2 | 2.7 | 22112333² | 58.1 | 58.0 | 4.2 |
| 21312213¹ | 60.9 | 60.6 | 1.1 | 21113333² | 58.1 | 61.0 | 4.1 |
| 21312332¹ | 60.8 | 59.9 | 1.3 | 22112233² | 57.0 | 58.7 | 5.2 |

¹predicted to be highly stable by linear regression;
²predicted to be stable by consensus analysis;
³activity on 2-phenoxyethanol is reported as total turnover number normalized to the most active parent protein, Al.

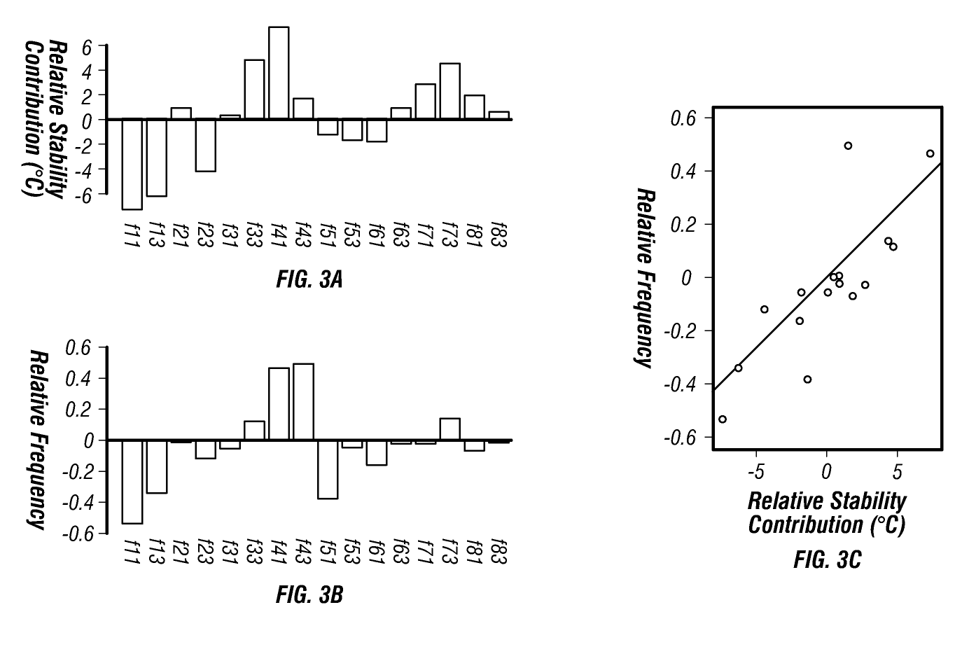

Consensus analysis of folded and unfolded sequences. Whether the multiple sequence alignment of the folded chimeras could be used to predict the stable sequences, similar to ‘consensus stabilization’ methods based on natural sequence alignments were analyzed. From the collection of folded chimeras and the estimates of fragment stability contributions, whether protein fragments that appear more often in the folded chimeras contribute more to protein stability can be assessed. The stability contributions of each fragment (relative to the least-stable parent, A2) derived from the regression analysis (FIG. 3a) were determined and the difference in frequency of each fragment (relative to A2) calculated in the set of 620 folded chimeras (FIG. 3b). These data reveal a strong positive relationship between relative stability contribution and relative fragment frequency (FIG. 3c).

To assess the predictive value of sequence-element frequencies more quantitatively, the stability of each chimera was calculated using the approach of Steipe et al. Assuming the frequency of a fragment at position i is exponentially related to its stability contribution and that these fragment contributions are additive, a total chimera consensus energy can be calculated from

$${ɛ}_{total} = {\sum }_{i }^{ } - ln {f}_{i} .$$

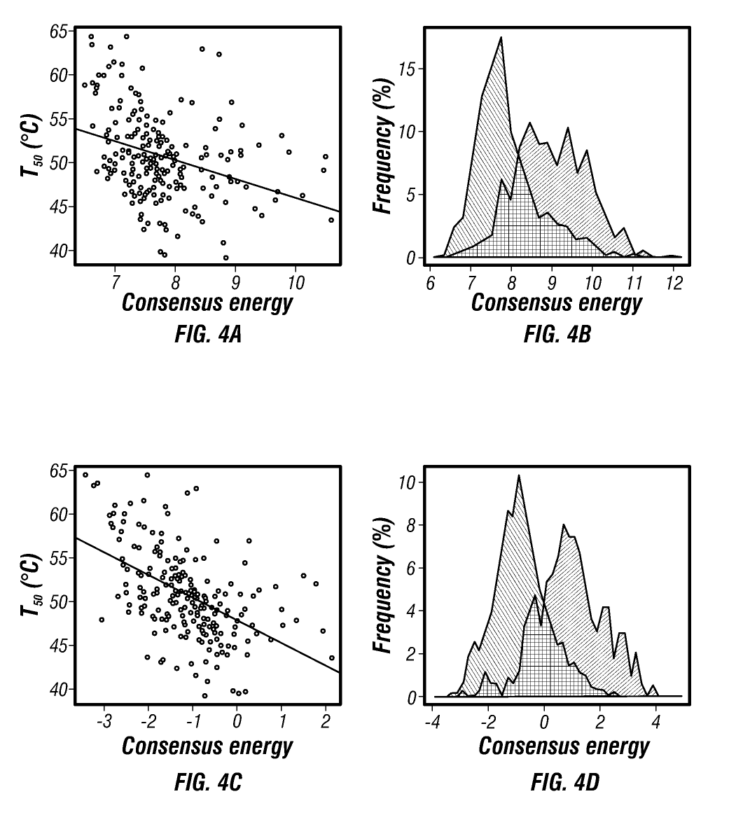

Lower consensus energies (based on the multiple sequence alignment (MSA) of 620 folded chimeras) are associated with higher T₅₀ values (FIG. 4a; Pearson r=0.34, P<10⁻⁵). Furthermore, folded proteins tend to have lower consensus energies than unfolded ones (FIG. 4b; Wilcoxon signed rank test P<<10⁻⁹).

A unique feature of the synthetic protein family is that it includes unfolded sequences, and those unfolded sequences contain useful information. Destabilizing effects were thought to follow the same pattern as stabilizing ones, such that the frequency of a fragment at position i in the unfolded population μᵢ predicts its destabilizing effect just as frequency in the folded population predicts its stabilizing effect. Revised chimera consensus energies

$${ɛ}_{total} = {\sum }_{i }^{ } - ln ( {f}_{i} / {u}_{i} ) ,$$

computed using fragment frequencies from the 620 folded and 335 unfolded sequences, significantly improved prediction of both stability (FIG. 4c; Pearson r=−0.53, P<10⁻¹⁵) and folding status (FIG. 4d). Incorporating the unfolded sequences in this way makes interpretation of the consensus energies straightforward: fragment with energies below zero appear more often in folded vs. unfolded proteins and tend to have stabilizing effects. It also helps control for small biases in chimera library construction or sampling which are the same for both multiple sequence alignments and which therefore cancel out in the calculation of consensus energy.

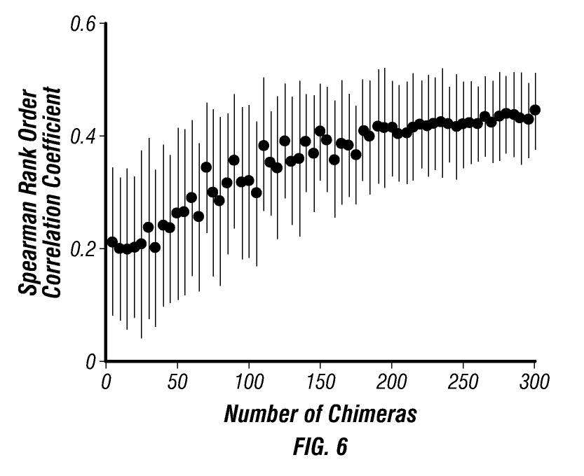

The tradeoff between the number of chimera sequences used to calculate the energies and the statistical error associated with ranking chimeras by consensus was calculated. A random subsets containing 5, 10, 15 . . . 300 sequences from 628 chimeras were selected at random from the SCHEMA library (a mixture of folded and unfolded chimeras) and calculated consensus energies for three parents and 205 chimeras with known T₅₀s. The Spearman rank correlation coefficient (rₛ) was then calculated between the consensus energy predictions and the measured T₅₀ values. This was repeated 50 times, and the average rₛ and standard deviation calculated for each sample size (FIG. 6). The average rank-order correlation coefficient is reliably above 0.4 (with standard deviations values less than 0.09) when 200 or more chimera sequences are used.

Protein stabilization by consensus. Having demonstrated that sequence and folding status alone can be used to make nontrivial predictions of relative stability, the most stable chimeras were then predicted. The total consensus energies of all 6,561 chimeras in the library were calculated; the 20 with the lowest consensus energies are listed in Table 4. Due to bias in the library construction, the data set of 955 chimeras has very few representatives of A2 at position 4, preventing accurate assessment of this fragment's thermostability contribution. Three sequences with this fragment were not constructed; the remaining seventeen were constructed. These chimeras are all highly stable, with T₅₀ values between 58.2° C. and 64.4° C. (Table 3). The sequence with consensus fragments at all eight positions (21312333) and therefore the lowest consensus energy is the “consensus sequence”, and should be the most stable chimera. Indeed, the consensus sequence has the highest measured stability among all 239 chimeras with known T₅₀ and is also the MTP predicted by the linear regression model.

TABLE 4
The 20 chimeras with lowest total consensus energies.

| Sequence | Consensus energy | Sequence | Consensus energy |
|---|---|---|---|
| 21312333 | −3.40 | 22312233 | −3.10 |
| 21312233 | −3.35 | 21322233 | −3.07 |
| 21112333 | −3.29 | 21313233 | −3.06 |
| 21212333 | −3.24 | 21312231 | −3.04 |
| 21112233 | −3.24 | 22112333 | −3.04 |
| 21212233 | −3.18 | 21122333 | −3.01 |
| 22312333 | −3.15 | 21113333 | −3.00 |
| 21322333 | −3.13 | 21112331 | −2.99 |
| 21313333 | −3.12 | 22212333 | −2.98 |
| 21312331 | −3.10 | 22112233 | −2.98 |

Stability predictions identify errors in sequencing. The stability predictions were found sufficiently accurate to identify both sequencing errors and point mutations in the chimeras. The sequences of P450 chimeras were originally determined in high throughput by DNA probe hybridization, which has a ˜3% error rate; small numbers of point mutations during library construction are also expected. Thus approximately 7 incorrect sequence readings are expected for the total set of 239 chimeras studied, and other sequences may have point mutations. 13 chimeras with prediction error of more than 4° C. from the original set of 190 chimeras whose T₅₀s were measured and analyzed by linear regression were resequences. Five either had incorrect sequences or contained point mutations (Table 5); these five chimeras were eliminated from the subsequent linear regression analysis to determine the model parameters in Table 2.

TABLE 5
Sequence Errors and Mutations Identified by Linear Regression

| | | | | Predicted | Predicted |
|---|---|---|---|---|---|
| | | | | T₅₀ (° C.) | T₅₀ (° C.) |
| Original | Correct | | Measured | (wrong | (correct |
| sequence | sequence | Mutation | T₅₀ (° C.) | sequence) | sequence) |
| 31312333 | 33332333 | no | 47.4 | 57.9 | 46.5 |
| 32333232 | 22333232 | no | 53.5 | 44.6 | 51.6 |
| 22131221 | 22131223 | no | 51.0 | 44.7 | 45.8 |
| 22212321 | same | P40L | 47.9 | 53.7 | — |
| 22312232 | same | Q354P | 53.4 | 58.1 | — |

Further work also showed that both the regression and consensus models do well enough to significantly increase the odds of identifying sequencing errors and mutations. From the initial high-throughput CO difference spectroscopy and probe hybridization sequencing analysis, chimeras 22313333, 21311311, and 22311333 had been labeled unfolded. All three, however, were predicted to be highly stable. Full sequencing showed that the original 22313333 construct was incomplete and missing some fragments. Re-constructed 22313333 was folded and very stable, with T₅₀=64.3° C. Similarly, the original 21311311 construct had an insertion, which after removal generated a chimera with T₅₀=61.0° C. Finally, 22311333 had two point mutations leading to two amino acid substitutions. After these mutations were corrected by site directed mutagenesis, 22311333 was shown to fold properly, with T₅₀=60.1° C.

Further characterization of stable cytochrome P450 chimeras. Important measures of stability for protein applications include the ability to withstand denaturing conditions, including elevated temperatures. An enzyme's half-life of (irreversible) inactivation (t_{1/2}) is commonly used to describe stability, and t_{1/2} often correlates with other stability measures. The t_{1/2} was measured at 57° C. for 13 stable chimeras and the three parents (Table 6). The results show that the increased stability can have a profound effect on half-life: while the most stable parent, A1, lost its ability to bind CO with a half-life of 15 min at this temperature, chimera 21312231 had a half -life of 1600 min, or more than 108 times greater. The MTP, chimera 21312333 also had a very long half-life, at 1550 min. T₅₀ has also been shown to correlate linearly with urea concentrations required for half-maximal denaturation for variants of CYP102A1. Thus it was expected that the stable P450 chimeras are also more tolerant to inactivation by denaturants.

TABLE 6
Half-lives of inactivation (t1/2) at 57° C. of three
parent proteins and 13 stabilized chimeric proteins.

| Sequence | t_{1/2} (min) |
|---|---|
| 11111111 | 15 |
| 22222222 | 0.36 |
| 33333333 | 0.86 |
| 21313333 | 350 |
| 22312331 | 170 |
| 21313233 | 160 |
| 21312331 | 110 |
| 21313231 | 930 |
| 22312233 | 400 |
| 22312231 | 140 |
| 21313331 | 390 |
| 21312231 | 1600 |
| 21312233 | 980 |
| 22312333 | 670 |
| 22313333 | 150 |
| 21312333 | 1550 |

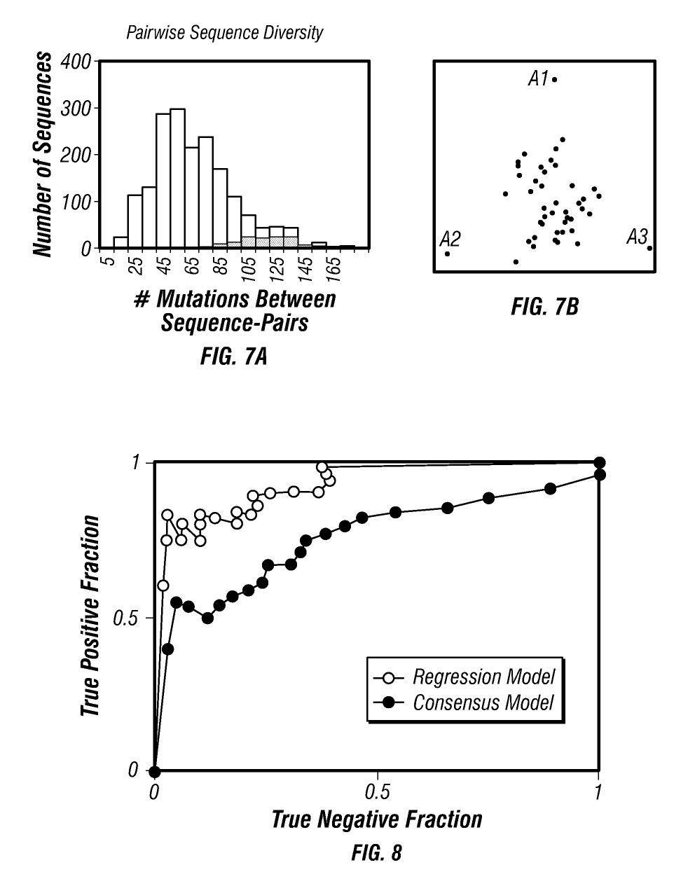

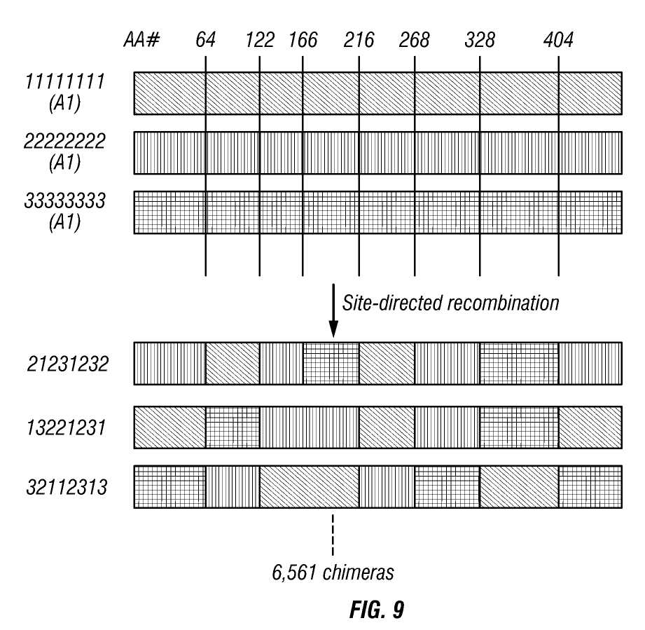

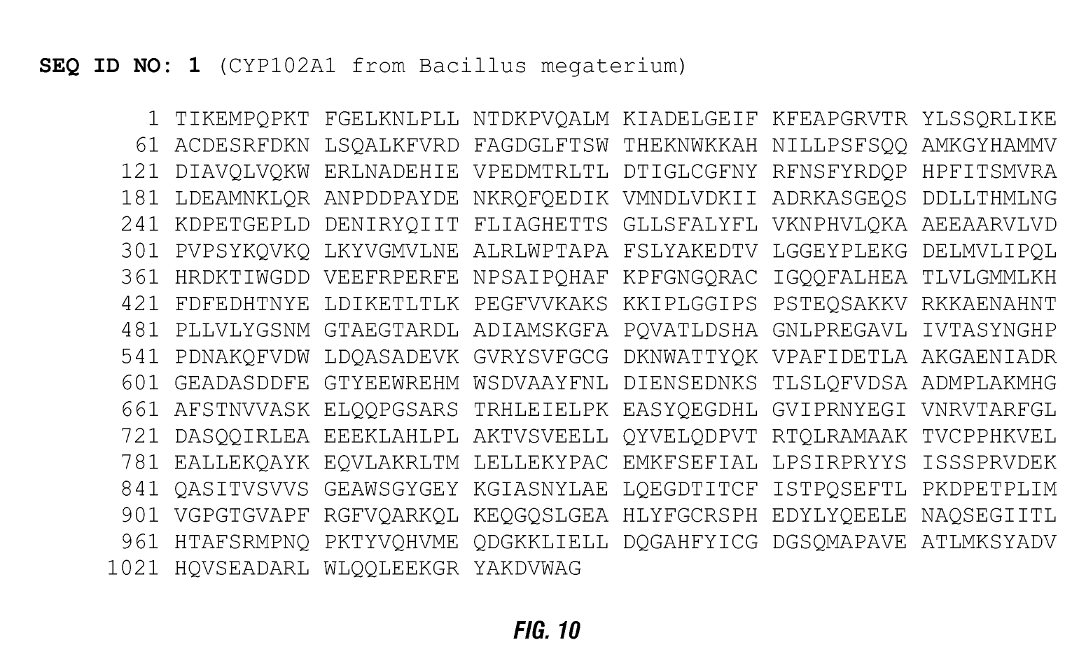

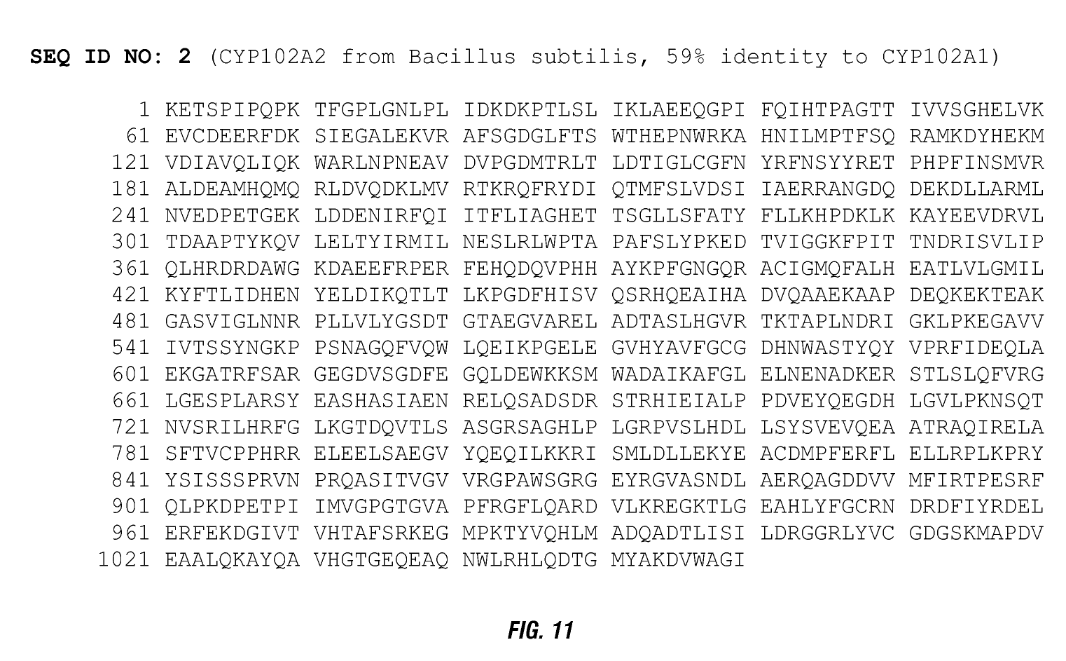

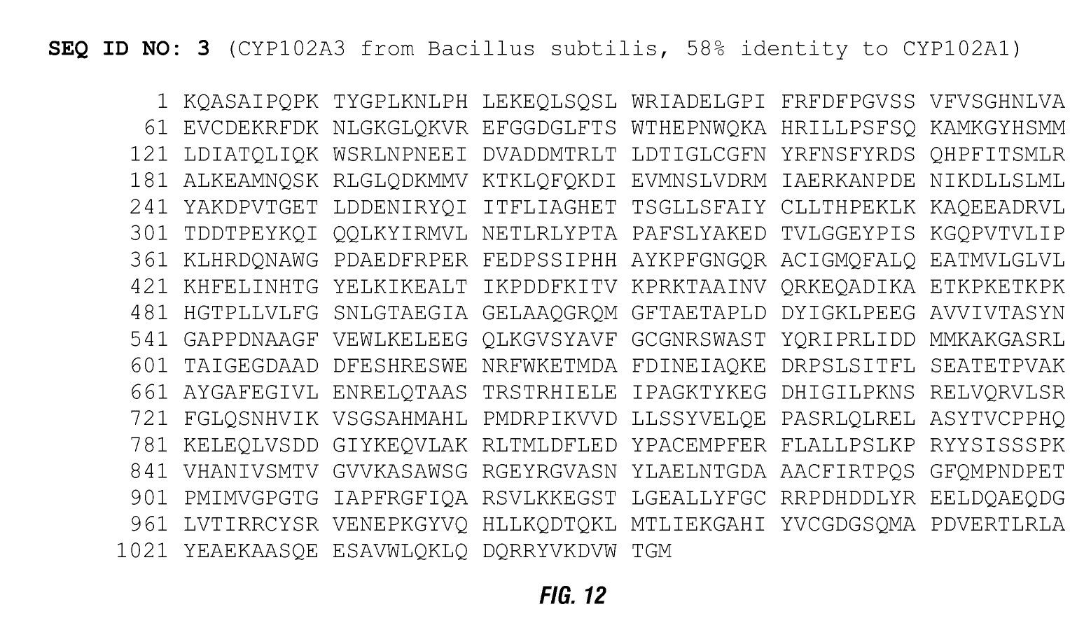

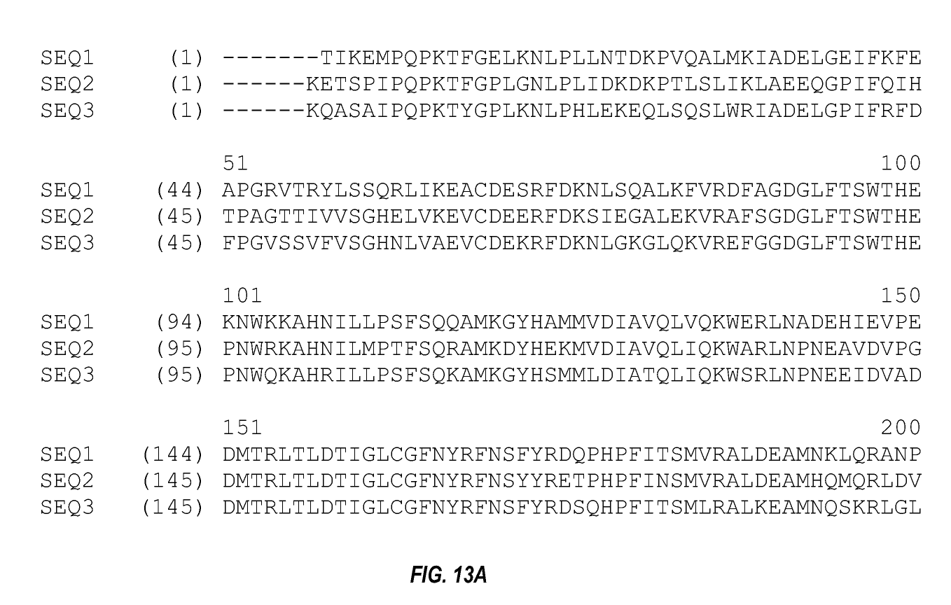

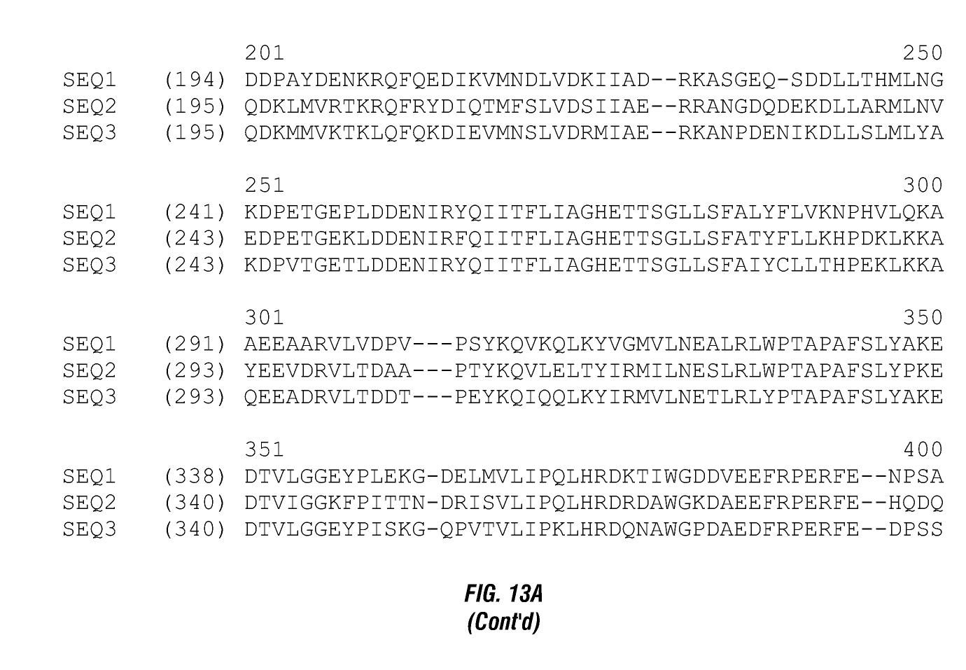

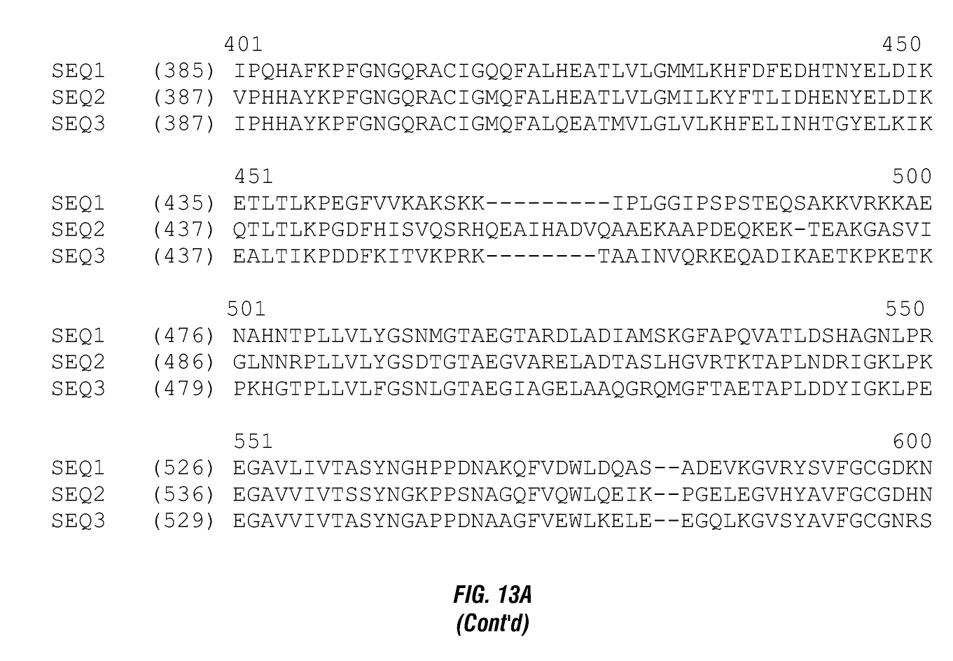

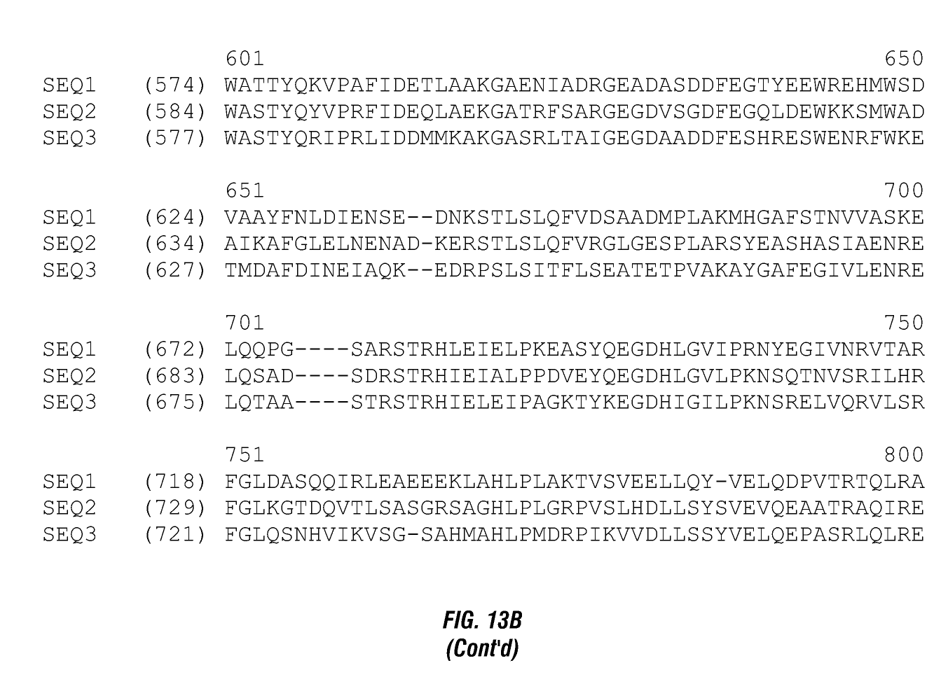

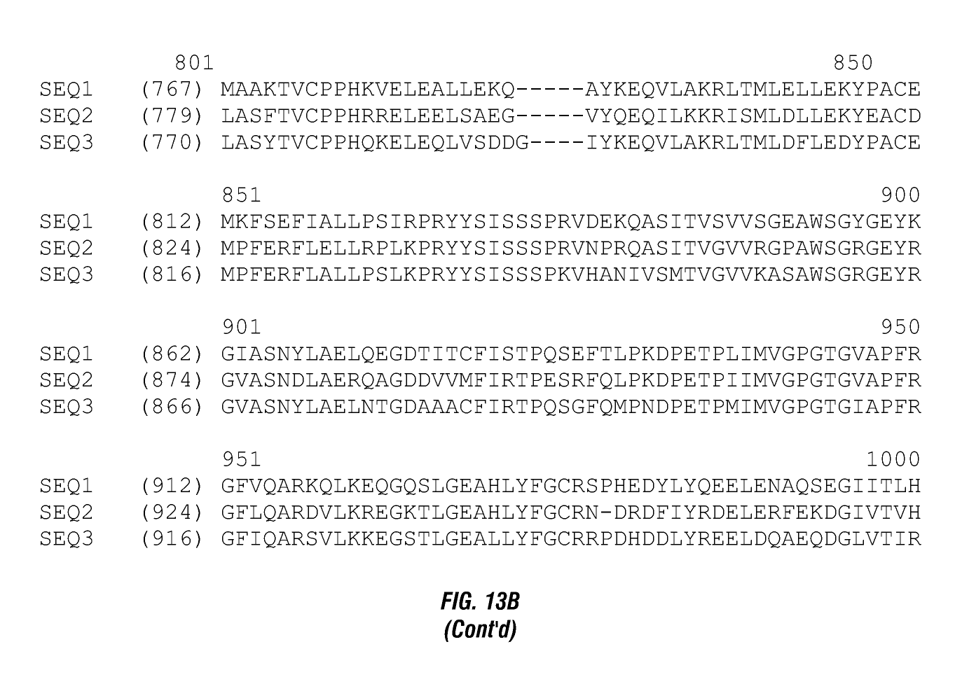

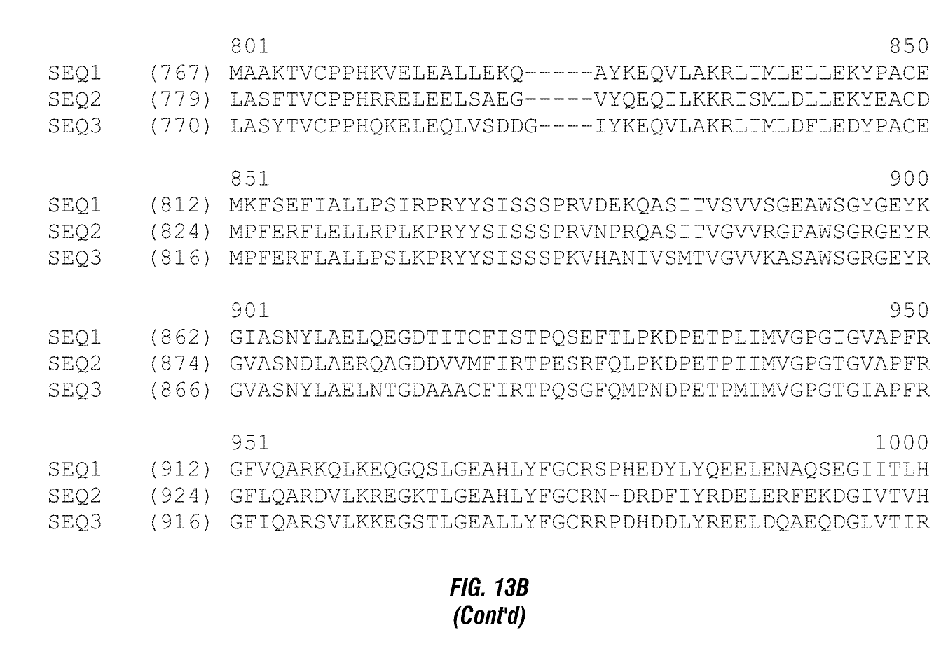

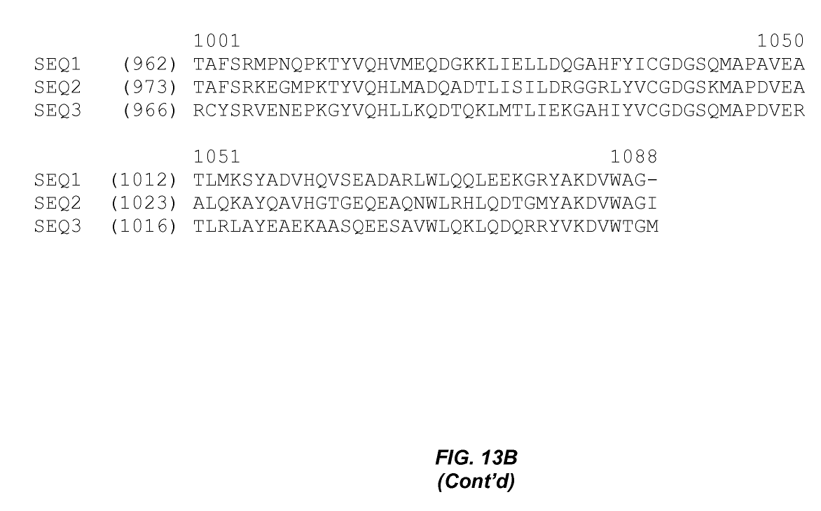

All 40 new chimeras were verified by full sequencing to eliminate any possibility that the enhanced thermostabilities were due to mutations, insertions or deletions. The 40 stable chimeras comprise a diverse family of sequences, differing from one another at 14 to 88 amino acid positions (49 on average) (FIG. 7). The distance to the closest parent is as high as 100 amino acids. The 40 chimeras thus comprise a family of properly folded, highly stable cytochrome P450s that exhibit considerable sequence novelty.

The activities of the stable chimeras were assessed in order to explore the relationship between activity and stability, and specifically to determine whether the increased stability came at the cost of catalytic function. The 40 thermostable chimeras and the three parents were assayed for peroxygenase activity on 2-phenoxyethanol, a substrate that is accepted by all three parent enzymes. All 40 chimeras were active on 2-phenoxyethanol (Table 3). Furthermore, many of them were more active than the most active parent (A1). One of the most stable enzymes (T₅₀=64.3° C.), chimera 22313333 was 9 times more active at room temperature than the most active parent. Consistent with observations from the extensive analysis of the substrate specificities of a sampling of the P450 chimeras, changes in sequence can have significant effects on relative activity. Chimera 21313333, for example, was also highly stable, but had less than half the activity of 2231333 on this nonnatural substrate (Table 3).

The protein expression levels of most of the thermostable chimeras were higher than those of the parent proteins. Most thermostable chimeras expressed well even without the inducing agent isopropyl-beta-D-thiogalactopyranoside (IPTG). Thus the family of 40 chimeric P450s reported here represents a set of well-expressed, highly stable, catalytically active enzymes with novel sequences.

1. DePristo, M. A., Weinreich, D. M. & Hartl, D. L. Missense meanderings in sequence space: A biophysical view of protein evolution. Nat. Rev. Genet. 6, 678-687 (2005).

2. Yue, P., Li, Z. L. & Moult, J. Loss of protein structure stability as a major causative factor in monogenic disease. J. Mol. Biol. 353, 459-473 (2005).

3. Bloom, J. D. et al. Thermodynamic prediction of protein neutrality. Proc. Nat. Acad. Sci. USA 102, 606-611 (2005).

4. Bloom, J. D., Labthavikul, S. T., Otey, C. R. & Arnold, F. H. Protein stability promotes evolvability Proc. Nat. Acad. Sci. USA 103, 5869-5874 (2006).

5. Drummond, D. A., Bloom, J. D., Adami, C., Wilke, C. O. & Arnold, F. H. Why highly expressed proteins evolve slowly. Proc. Nat. Acad. Sci. USA 102, 14338-14343 (2005).

6. Niehaus, F., Bertoldo, C., Kahler, M. & Antranikian, G. Extremophiles as a source of novel enzymes for industrial application. Appl. Microbiol. Biot. 51, 711-729 (1999).

7. Zeikus, J. G., Vieille, C. & Savchenko, A. Thermozymes: biotechnology and structure-function relationships. Extremophiles 2, 179-183 (1998).

8. Guengerich, F. P. Cytochrome P450 enzymes in the generation of commercial products. Nat. Rev. Drug Discov. 1, 359-366 (2002).

9. Landwehr, M. et al. Enantioselective alpha-hydroxylation of 2-arylacetic acid derivatives and buspirone catalyzed by engineered cytochrome P45OBM-3. J. Am. Chem. Soc. 128, 6058-6059 (2006).

10. Otey, C. R., Bandara, G., Lalonde, J., Takahashi, K. & Arnold, F. H. Preparation of human metabolites of propranolol using laboratory-evolved bacterial cytochromes P450. Biotechnol. Bioeng. 93, 494-499 (2006).

11. Urlacher, V. B. & Eiben, S. Cytochrome P450 monooxygenases: perspectives for synthetic application. Trends Biotechnol. 24, 324-330 (2006).

12. van Vugt-Lussenburg, B. M. A. et al. Heterotropic and homotropic cooperativity by a drug-metabolising mutant of cytochrome P450BM3. Biochem. Bioph. Res. Comm. 346, 810-818 (2006).

13. Otey, C. R. et al. Structure-guided recombination creates an artificial family of cytochromes P450. PLOS Biol. 4, e112 (2006).

14. Dietterich, T. G. Approximate statistical tests for comparing supervised classification learning algorithms. Neural Comput. 10, 1895-1923 (1998).

15. Fox, R. et al. Optimizing the search algorithm for protein engineering by directed evolution. Protein Eng. 16, 589-597 (2003).

16. Amin, N. et al. Construction of stabilized proteins by combinatorial consensus mutagenesis. Protein Eng. Des. Sel. 17, 787-793 (2004).

17. Lehmann, M. et al. The consensus concept for thermostability engineering of proteins: further proof of concept. Protein Eng. 15, 403-411 (2002).

18. Steipe, B., Schiller, B., Pluckthun, A. & Steinbacher, S. Sequence statistics reliably predict stabilizing mutations in a protein domain. J. Mol. Biol. 240, 188-192 (1994).

19. Joern, J. M., Meinhold, P. & Arnold, F. H. Analysis of shuffled gene libraries. J. Mol. Biol. 316, 643-656 (2002).

20. Johannes, T. W., Woodyer, R. D., & Zhao, H. M. Directed evolution of a thermostable phosphite dehydrogenase for NAD(P)H regeneration. Appl. Environ. Microb. 71, 5728-5734 (2005)

21. Landwehr, M., Carbone, M., Otey, C. R., Li, Y. & Arnold, F. H. Diversification of catalytic function in a synthetic family of chimeric cytochrome P450s. Chem. Biol. In press (2007).

22. Somero, G. N. Proteins and temperature. Annu. Rev. Physiol. 57, 43-68 (1995).

23. Arnold, F. H., Wintrode, P. L., Miyazaki, K. & Gershenson, A. How enzymes adapt: lessons from directed evolution. Trends Biochem. Sci. 26, 100-106 (2001).

24. Taverna, D. M. & Goldstein, R. A. Why are proteins marginally stable? Proteins 46, 105-109 (2002).

25. Bloom, J. D., Raval, A. & Wilke , C. O. Thermodynamics of neutral protein evolution Genetics 175, 255-266 (2007).

26. Serrano, L., Day, A.G. & Fersht, A. R. Step-wise mutation of barnase to binase—a procedure for engineering increased stability of proteins and an experimental-analysis of the evolution of protein stability. J. Mol. Biol. 233, 305-312 (1993).

27. Giver, L., Gershenson, A., Freskgard, P. O. & Arnold, F. H. Directed evolution of a thermostable esterase. Proc. Nat. Acad. Sci. USA 95, 12809-12813 (1998).

What is claimed is:

1. A polypeptide selected from the group consisting of:
(a) a polypeptide comprising sequences from CYP102A1 (“1”), A2 (“2”) and A3 (“3”) having the general formula 21122333, from N-terminus to C-terminus;
(b) a polypeptide comprising sequences from CYP102A1 (“1”), A2 (“2”) and A3 (“3”) having the general formula 21322233, from N-terminus to C-terminus; and
(c) a polypeptide comprising sequences from CYP102A1 (“1”), A2 (“2”) and A3 (“3”) having the general formula 22313333, from N-terminus to C-terminus,
and wherein the polypeptide has a CO-binding peak at 450 nm.

2. The polypeptide of claim 1, wherein the polypeptide comprises:
a first peptide segment comprising about 64 to 68 amino acids having at the C-terminus of the first peptide segment a sequence E(S or E or K)RFD (SEQ ID NO:4),
a second peptide segment comprising about 56 to 60 amino acids having at the C-terminus of the second peptide segment a sequence K(G or D)YH(A or E or S) (SEQ ID NO:5),
a third peptide segment comprising about 42 to 46 amino acids having at the C-terminus of the third peptide segment a sequence GFNYR (SEQ ID NO:6),
a fourth peptide segment comprising about 48 to 52 amino acid having at the C-terminus of the fourth peptide segment a sequence (D or S)LVD(K or S or R) (SEQ ID NO:7),
a fifth peptide segment comprising about 50 to 54 amino acids having at the C-terminus of the fifth peptide segment a sequence HETTS (SEQ ID NO:8),
a sixth peptide segment comprising about 58 to 62 amino acids having at the C-terminus of the sixth peptide segment a sequence PTAPA (SEQ ID NO:9),
a seventh peptide segment comprising about 74 to 78 amino acids having at the C-terminus of the seventh peptide segment a sequence G(Q or M)QFA (SEQ ID NO:10),
an eighth peptide segment comprising a sequence extending from the C-terminus of the seventh peptide segment to the C-terminus of the heme domain of a P450 BM3 of SEQ ID NO:1, 2, or 3, or the C-terminus of a P450 BM3 of SEQ ID NO:1, 2, or 3,
and wherein the polypeptide has a CO-binding peak at 450 nm.

3. The polypeptide of claim 1 or 2, wherein the polypeptide of (a) comprises from N-terminus to C-terminus SEQ ID NO:2 from amino acid residue 1 to about x1, SEQ ID NO:1 from about amino acid residue x1 to about x2, SEQ ID NO:1 from about amino acid residue x2 to about x3, SEQ ID NO:2 from about amino acid residue x3 to about x4, SEQ ID NO:2 from about amino acid residue x4 to about x5, SEQ ID NO:3 from about amino acid residue x5 to about x6, SEQ ID NO:3 from about amino acid residue x6 to about x7, SEQ ID NO:3 from about amino acid residue x7 to about x8;
wherein x1 comprises residue 63, 64, 65, 66, or 67 of SEQ ID NO:2; x2 comprises residue 120, 121, 122, 123, or 124 of SEQ ID NO:1; x3 comprises residue 164, 165, 166, 167 or 168 of SEQ ID NO:1; x4 comprises residue 215, 216, 217, 218 or 219 of SEQ ID NO:2; x5 comprises residue 268, 269, 270, 271, or 272 of SEQ ID NO:2; x6 comprises residue 328, 329, 330, 331, or 332 of SEQ ID NO:3; x7 comprises residue 404, 405, 406, 407, or 408 of SEQ ID NO:3; and x8 comprises a residue corresponding to the C-terminus of the heme domain of CYP102A3 or the C-terminus of CYP102A3 of SEQ ID NO:3,
and wherein the polypeptide has a CO-binding peak at 450 nm.

4. The polypeptide of claim 1 or 2, wherein the polypeptide of (b) comprises from N-terminus to C-terminus SEQ ID NO:2 from amino acid residue 1 to about x1, SEQ ID NO:1 from about amino acid residue x1 to about x2, SEQ ID NO:3 from about amino acid residue x2 to about x3, SEQ ID NO:2 from about amino acid residue x3 to about x4, SEQ ID NO:2 from about amino acid residue x4 to about x5, SEQ ID NO:2 from about amino acid residue x5 to about x6, SEQ ID NO:3 from about amino acid residue x6 to about x7, SEQ ID NO:3 from about amino acid residue x7 to about x8;
wherein x1 comprises residue 63, 64, 65, 66, or 67 of SEQ ID NO:2; x2 comprises residue 120, 121, 122, 123, or 124 of SEQ ID NO:1; x3 comprises residue 164, 165, 166, 167 or 168 of SEQ ID NO:3; x4 comprises residue 215, 216, 217, 218 or 219 of SEQ ID NO:2; x5 comprises residue 268, 269, 270, 271, or 272 of SEQ ID NO:2; x6 comprises residue 328, 329, 330, 331, or 332 of SEQ ID NO:2; x7 comprises residue 404, 405, 406, 407, or 408 of SEQ ID NO:3; and x8 comprises a residue corresponding to the C-terminus of the heme domain of CYP102A3 or the C-terminus of CYP102A3 of SEQ ID NO:3,
and wherein the polypeptide has a CO-binding peak at 450 nm.

5. The polypeptide of claim 1 or 2, wherein the polypeptide of (c) comprises from N-terminus to C-terminus SEQ ID NO:2 from amino acid residue 1 to about x1, SEQ ID NO:2 from about amino acid residue x1 to about x2, SEQ ID NO:3 from about amino acid residue x2 to about x3, SEQ ID NO:1 from about amino acid residue x3 to about x4, SEQ ID NO:3 from about amino acid residue x4 to about x5, SEQ ID NO:3 from about amino acid residue x5 to about x6, SEQ ID NO:3 from about amino acid residue x6 to about x7, SEQ ID NO:3 from about amino acid residue x7 to about x8;
wherein x1 comprises residue 63, 64, 65, 66, or 67 of SEQ ID NO:2; x2 comprises residue 121, 122, 123, 124, or 125 of SEQ ID NO:2; x3 comprises residue 164, 165, 166, 167 or 168 of SEQ ID NO:3; x4 comprises residue 214, 215, 216, 217 or 218 of SEQ ID NO:1; x5 comprises residue 268, 269, 270, 271, or 272 of SEQ ID NO:3; x6 comprises residue 328, 329, 330, 331, or 332 of SEQ ID NO:3; x7 comprises residue 404, 405, 406, 407, or 408 of SEQ ID NO:3; and x8 comprises a residue corresponding to the C-terminus of the heme domain of CYP102A3 or the C-terminus of CYP102A3 of SEQ ID NO:3,
and wherein the polypeptide has a CO-binding peak at 450 nm.

6. A polynucleotide encoding a polypeptide of claim 1.

7. A vector comprising a polynucleotide of claim 6.

8. A host cell comprising the vector of claim 7.

9. A host cell comprising the polynucleotide of claim 7.

10. An enzyme extract comprising a polypeptide produced from the host cell of claim 8 or 9.


SEQUENCE LISTING (10 sequences)

SEQ ID NO: 1 - protein, 1048 aa, Bacillus megaterium
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 2 - protein, 1060 aa, Bacillus subtilis
KETSPIPQPKTFGPLGNLPLIDKDKPTLSLIKLAEEQGPIFQIHTPAGTTIVVSGHELVKEVCDEERFDKSIEGALEKVRAFSGDGLFTSWTHEPNWRKAHNILMPTFSQRAMKDYHEKMVDIAVQLIQKWARLNPNEAVDVPGDMTRLTLDTIGLCGFNYRFNSYYRETPHPFINSMVRALDEAMHQMQRLDVQDKLMVRTKRQFRYDIQTMFSLVDSIIAERRANGDQDEKDLLARMLNVEDPETGEKLDDENIRFQIITFLIAGHETTSGLLSFATYFLLKHPDKLKKAYEEVDRVLTDAAPTYKQVLELTYIRMILNESLRLWPTAPAFSLYPKEDTVIGGKFPITTNDRISVLIPQLHRDRDAWGKDAEEFRPERFEHQDQVPHHAYKPFGNGQRACIGMQFALHEATLVLGMILKYFTLIDHENYELDIKQTLTLKPGDFHISVQSRHQEAIHADVQAAEKAAPDEQKEKTEAKGASVIGLNNRPLLVLYGSDTGTAEGVARELADTASLHGVRTKTAPLNDRIGKLPKEGAVVIVTSSYNGKPPSNAGQFVQWLQEIKPGELEGVHYAVFGCGDHNWASTYQYVPRFIDEQLAEKGATRFSARGEGDVSGDFEGQLDEWKKSMWADAIKAFGLELNENADKERSTLSLQFVRGLGESPLARSYEASHASIAENRELQSADSDRSTRHIEIALPPDVEYQEGDHLGVLPKNSQTNVSRILHRFGLKGTDQVTLSASGRSAGHLPLGRPVSLHDLLSYSVEVQEAATRAQIRELASFTVCPPHRRELEELSAEGVYQEQILKKRISMLDLLEKYEACDMPFERFLELLRPLKPRYYSISSSPRVNPRQASITVGVVRGPAWSGRGEYRGVASNDLAERQAGDDVVMFIRTPESRFQLPKDPETPIIMVGPGTGVAPFRGFLQARDVLKREGKTLGEAHLYFGCRNDRDFIYRDELERFEKDGIVTVHTAFSRKEGMPKTYVQHLMADQADTLISILDRGGRLYVCGDGSKMAPDVEAALQKAYQAVHGTGEQEAQNWLRHLQDTGMYAKDVWAGI

SEQ ID NO: 3 - protein, 1053 aa, Bacillus subtilis
KQASAIPQPKTYGPLKNLPHLEKEQLSQSLWRIADELGPIFRFDFPGVSSVFVSGHNLVAEVCDEKRFDKNLGKGLQKVREFGGDGLFTSWTHEPNWQKAHRILLPSFSQKAMKGYHSMMLDIATQLIQKWSRLNPNEEIDVADDMTRLTLDTIGLCGFNYRFNSFYRDSQHPFITSMLRALKEAMNQSKRLGLQDKMMVKTKLQFQKDIEVMNSLVDRMIAERKANPDENIKDLLSLMLYAKDPVTGETLDDENIRYQIITFLIAGHETTSGLLSFAIYCLLTHPEKLKKAQEEADRVLTDDTPEYKQIQQLKYIRMVLNETLRLYPTAPAFSLYAKEDTVLGGEYPISKGQPVTVLIPKLHRDQNAWGPDAEDFRPERFEDPSSIPHHAYKPFGNGQRACIGMQFALQEATMVLGLVLKHFELINHTGYELKIKEALTIKPDDFKITVKPRKTAAINVQRKEQADIKAETKPKETKPKHGTPLLVLFGSNLGTAEGIAGELAAQGRQMGFTAETAPLDDYIGKLPEEGAVVIVTASYNGAPPDNAAGFVEWLKELEEGQLKGVSYAVFGCGNRSWASTYQRIPRLIDDMMKAKGASRLTAIGEGDAADDFESHRESWENRFWKETMDAFDINEIAQKEDRPSLSITFLSEATETPVAKAYGAFEGIVLENRELQTAASTRSTRHIELEIPAGKTYKEGDHIGILPKNSRELVQRVLSRFGLQSNHVIKVSGSAHMAHLPMDRPIKVVDLLSSYVELQEPASRLQLRELASYTVCPPHQKELEQLVSDDGIYKEQVLAKRLTMLDFLEDYPACEMPFERFLALLPSLKPRYYSISSSPKVHANIVSMTVGVVKASAWSGRGEYRGVASNYLAELNTGDAAACFIRTPQSGFQMPNDPETPMIMVGPGTGIAPFRGFIQARSVLKKEGSTLGEALLYFGCRRPDHDDLYREELDQAEQDGLVTIRRCYSRVENEPKGYVQHLLKQDTQKLMTLIEKGAHIYVCGDGSQMAPDVERTLRLAYEAEKAASQEESAVWLQKLQDQRRYVKDVWTGM

SEQ ID NO: 4 - protein, 5 aa, Artificial sequence
  Consensus peptide sequence
EXRFD

SEQ ID NO: 5 - protein, 5 aa, Artificial Sequence
  Consensus peptide sequence
KXYHX

SEQ ID NO: 6 - protein, 5 aa, Artificial Sequence
  Consensus peptide sequence
GFNYR

SEQ ID NO: 7 - protein, 5 aa, Artificial sequence
  Consensus peptide sequence
XLVDX

SEQ ID NO: 8 - protein, 5 aa, Artificial sequence
  Consensus peptide sequence
HETTS

SEQ ID NO: 9 - protein, 5 aa, Artificial sequence
  Consensus peptide sequence
PTAPA

SEQ ID NO: 10 - protein, 5 aa, Artificial sequence
  Consensus peptide sequence
GXQFA